(19) United States
(12) Patent Application Publication
(10) Pub. No.: US 2026/0058014 A1
(43) Pub. Date: Feb. 26, 2026

(54) CHARACTERIZATION OF TUMOR HETEROGENEITY AS A PROGNOSTIC BIOMARKER

(71) Applicant: Foundation Medicine, Inc., Boston, MA (US)

(72) Inventors: Karthikeyan MURUGESAN, Boston, MA (US); Meagan Kathleen MONTESION, Somerville, MA (US); David FABRIZIO, Cambridge, MA (US); Khaled A. TOLBA, Portland, OR (US); Garrett M. FRAMPTON, Somerville, MA (US)

(73) Assignee: Foundation Medicine, Inc., Boston, MA (US)

(21) Appl. No.: 18/847,591

(22) PCT Filed: Mar. 16, 2023
(86) PCT No.: PCT/US2023/064598

§ 371 Date: Sep. 16, 2024

Related U.S. Application Data
(60) Provisional application No. 63/323,017, filed on Mar. 23, 2022.

(51) Int. Cl. G16H 50/20; A61K 31/519; C12Q 1/6855; C12Q 1/6874; G16B 20/20; G16H 15/00; G16H 20/10; G16H 50/70

(52) U.S. Cl. CPC G16H 50/20; A61K 31/519; C12Q 1/6855; C12Q 1/6874; G16B 20/20; G16H 15/00; G16H 20/10; G16H 50/70; C12Q 2600/106

(57) ABSTRACT

Methods for using tumor heterogeneity score as a biomarker to guide treatment and/or patient monitoring decisions are described. The methods may comprise, for example, acquiring knowledge of a driver mutation in a sample obtained from a subject; identifying one or more variants present in the sample; determining a tumor heterogeneity score for the sample based on the one or more variants; and comparing the tumor heterogeneity score for the sample to a threshold tumor heterogeneity score, where if the tumor heterogeneity score is less than or equal to the threshold tumor heterogeneity score, the subject is identified for treatment with a first anti¬cancer agent, and where if the tumor heterogeneity score is greater than the threshold tumor heterogeneity score, the subject is identified for treatment with a second anti-cancer agent.

### CROSS-REFERENCE TO RELATED APPLICATIONS

This application claims the priority benefit of U.S. Provisional Patent Application Ser. No. 63/323,017, filed on Mar. 23, 2022, the contents of which are incorporated herein by reference in their entirety.

### FIELD OF THE INVENTION

The present disclosure relates generally to methods and systems for analyzing genomic profiling data, and more specifically to methods and systems for using genomic profiling data for a patient to determine a tumor heterogeneity score that can serve as a prognostic biomarker for estimating the duration of clinical benefit for a selected disease treatment for the patient.

### BACKGROUND

Various biomarkers have been reported in the biomedical literature to be predictive of patient response to a targeted therapy or immunotherapy for a given disease (e.g., cancer). However, there is an unmet clinical need for prognostic biomarkers that are capable of predicting the duration of therapeutic response for an individual patient and a selected treatment. Duration of response (DoR) is the length of time that a given disease (e.g., a cancer) continues to respond to a treatment without the cancer growing or spreading, and can vary according to disease type, selected treatment, and individual patient. Healthcare providers often face a multitude of first line (1L) and second line (2L) treatment options, and would thus benefit from having access to a prognostic biomarker (e.g., based on genomic tumor heterogeneity) that helps to guide the decision-making for patient treatment selection and follow-up care—particularly for combination therapies used as a multi-pronged approach to treat tumors that are genetically heterogeneous.

### BRIEF SUMMARY OF THE INVENTION

Disclosed herein are methods and systems for determining a tumor heterogeneity measure (e.g., a tumor heterogeneity score) based on genomic data for an individual patient that is predictive of the estimated duration of the individual patient's response to a selected treatment for a given disease, e.g., a cancer. Also disclosed are methods for selecting a treatment and for treating a patient based on the tumor heterogeneity score. The disclosed methods have the potential to improve healthcare outcomes for patients (e.g., cancer patients) by providing healthcare providers with a better decision-making tool for guiding 1L and 2L treatment selection and for making recommendations for post-treatment patient monitoring. The disclosed methods for determining a tumor heterogeneity score may also for provide patients with a better understanding of their own prognosis.

Disclosed herein are methods comprising: providing a plurality of nucleic acid molecules obtained from a sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain sequence read data that represents the captured nucleic acid molecules; identifying a presence or absence of one or more variants in the sample based on the sequence read data; identifying a driver mutation in the sample based on the sequence read data; determining a tumor heterogeneity score for the sample based on the one or more variants identified as present in the sample; and comparing the tumor heterogeneity score to a threshold tumor heterogeneity score, wherein if the tumor heterogeneity score is less than or equal to the threshold tumor heterogeneity score, the subject is identified for treatment with a first anti-cancer agent selected at least in part based on the identified driver mutation, and wherein if the tumor heterogeneity score is greater than the threshold tumor heterogeneity score, the subject is identified for treatment with a second anti-cancer agent selected at least in part based on the identified driver mutation.

In some embodiments, the method further comprises determining a clonality metric for the driver mutation, wherein if the tumor heterogeneity score is less than or equal to the threshold tumor heterogeneity score and the clonality metric is greater than or equal to a threshold clonality metric, the subject is identified for treatment with the first anti-cancer agent. In some embodiments, the method further comprises determining a clonality metric for the driver mutation, wherein if the tumor heterogeneity score is greater than the threshold tumor heterogeneity score and the clonality metric is less than a threshold clonality metric, the subject is identified for treatment with a second anti-cancer agent. In some embodiments, if the tumor heterogeneity score is greater than the threshold tumor heterogeneity score, the subject is identified for tumor monitoring. In some embodiments, the subject is suspected of having or is determined to have a cancer. In some embodiments, the method further comprises obtaining the sample from the subject. In some embodiments, the sample comprises a tissue biopsy sample, a liquid biopsy sample, or a normal control. In some embodiments, the sample is a liquid biopsy sample and comprises blood, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof. In some embodiments, the plurality of nucleic acid molecules comprises a mixture of tumor nucleic acid molecules and non-tumor nucleic acid molecules. In some embodiments, the tumor nucleic acid molecules are derived from a tumor portion of a heterogeneous tissue biopsy sample, and the non-tumor nucleic acid molecules are derived from a normal portion of the heterogeneous tissue biopsy sample. In some embodiments, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample. In some embodiments, the one or more adapters comprise amplification primers, flow cell adaptor sequences, substrate adapter sequences, or sample index sequences. In some embodiments, the captured nucleic acid molecules are captured from the amplified nucleic acid molecules by hybridization to one or more bait molecules. In some embodiments, the one or more bait molecules comprise one or more nucleic acid molecules, each comprising a region that is complementary to a region of a captured nucleic acid molecule. In some embodiments, amplifying nucleic acid molecules comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or Sanger sequencing technique. In some embodiments, the sequencing comprises massively parallel sequencing, and the massively parallel sequencing technique comprises next generation sequencing (NGS). In some embodiments, the sequencer comprises a next generation sequencer. In some embodiments, the method further comprises generating, by the one or more processors, a report comprising a tumor heterogeneity score for the subject or a clonality metric for a driver mutation of the disease present in the sequence read data of the subject. In some embodiments, the method further comprises transmitting the report to a healthcare provider. In some embodiments, the report is transmitted via a computer network or a peer-to-peer connection.

Disclosed herein are methods for identifying a subject having a cancer for treatment with an anti-cancer agent comprising: acquiring knowledge of a driver mutation in a sample obtained from the subject; identifying one or more variants present in the sample; determining a tumor heterogeneity score for the sample based on the one or more variants; and comparing the tumor heterogeneity score for the sample to a threshold tumor heterogeneity score, wherein if the tumor heterogeneity score is less than or equal to the threshold tumor heterogeneity score, the subject is identified for treatment with a first anti-cancer agent selected at least in part based on the knowledge of the driver mutation, and wherein if the tumor heterogeneity score is greater than the threshold tumor heterogeneity score, the subject is identified for treatment with a second anti-cancer agent selected at least in part based on the knowledge of the driver mutation.

Also disclosed herein are methods of selecting a treatment for a subject having a cancer, the method comprising: acquiring knowledge of a driver mutation in a sample obtained from the subject; determining a tumor heterogeneity score for the sample obtained from the subject; and comparing the tumor heterogeneity score for the sample to a threshold tumor heterogeneity score, wherein if the tumor heterogeneity score that is less than or equal to the threshold tumor heterogeneity score, the subject is identified as one who may benefit from treatment with a first anti-cancer treatment selected at least in part based on the knowledge of the driver mutation; and wherein if the tumor heterogeneity score is greater than the threshold tumor heterogeneity score, the subject is identified as one who may benefit from treatment with a second anti-cancer treatment selected at least in part based on the knowledge of the driver mutation.

In some embodiments, the method further comprises determining a clonality metric for the driver mutation, wherein if the tumor heterogeneity score is less than or equal to the threshold tumor heterogeneity score and the clonality metric is greater than or equal to a threshold clonality metric, the subject is identified for treatment with the first anti-cancer therapy or as one who may benefit from treatment with the first anti-cancer treatment. In some embodiments, the method further comprises determining a clonality metric for the driver mutation, wherein if the tumor heterogeneity score is greater than the threshold tumor heterogeneity score and the clonality metric is less than a threshold clonality metric, the subject is identified for treatment with a second anti-cancer therapy or as one who may benefit from treatment with the second anti-cancer treatment. In some embodiments, if the tumor heterogeneity score is greater than the threshold tumor heterogeneity score, the subject is identified for tumor monitoring. In some embodiments, the tumor monitoring comprises follow-up tumor sequencing within 3, 6, 9, or 12 months. In some embodiments, the tumor monitoring comprises having a CT scan of the subject performed within 3, 6, 9, or 12 months.

Disclosed herein are methods of identifying one or more treatment options for a subject having a cancer, the method comprising: a) acquiring knowledge of a driver mutation in a sample obtained from the subject; b) determining a tumor heterogeneity score for the sample obtained from the subject; and c) generating a report comprising one or more treatment options identified for the subject that are selected at least in part based on the knowledge of the driver mutation, wherein a tumor heterogeneity score that is less than or equal to a corresponding threshold tumor heterogeneity score identifies the subject as one who may benefit from that treatment option.

In some embodiments, the method further comprises determining a clonality metric for the driver mutation and generating the report comprising one or more treatment options identified for the subject, wherein a tumor heterogeneity score that is less than or equal to a corresponding threshold tumor heterogeneity score for each of one or more candidate treatment options and a clonality metric that is greater than or equal to a threshold clonality metric for each of the one or more candidate treatment options identifies the subject as one who may benefit from that treatment option. In some embodiments, if the tumor heterogeneity score is greater than the threshold tumor heterogeneity score, the subject is identified for tumor monitoring.

Disclosed herein are methods of stratifying a subject with a cancer for treatment comprising: acquiring knowledge of a driver mutation in a sample obtained from the subject; determining a tumor heterogeneity score for the sample obtained from the subject; and a) if the tumor heterogeneity score is less than or equal to a threshold tumor heterogeneity score, identifying the subject as a candidate for receiving a first anti-cancer therapy selected at least in part based on the knowledge of the driver mutation; or b) if the tumor heterogeneity score is greater than the threshold tumor heterogeneity score, identifying the subject as a candidate for receiving a second anti-cancer therapy selected at least in part based on the knowledge of the driver mutation.

In some embodiments, the method further comprises determining a clonality metric for the driver mutation; and c) if the tumor heterogeneity score is less than or equal to a threshold tumor heterogeneity score for a first therapy and the clonality metric is greater than or equal to a threshold clonality metric for the first anti-cancer therapy, identifying the subject as a candidate for receiving the first anti-cancer therapy; or d) if the tumor heterogeneity score is greater than the threshold tumor heterogeneity score for the first therapy and the clonality metric is less than the threshold clonality metric for the first anti-cancer therapy, identifying the subject as a candidate for receiving a second anti-cancer therapy. In some embodiments, if the tumor heterogeneity score is greater than the threshold tumor heterogeneity score, the subject is identified for tumor monitoring.

Disclosed herein are methods for treating a subject having a cancer, the method comprising: a) acquiring knowledge of a driver mutation in a sample obtained from the subject; b) determining a tumor heterogeneity score for the sample obtained from the subject; and c) treating the subject with a first anti-cancer therapy selected at least in part based on the knowledge of the driver mutation if the tumor heterogeneity score is less than or equal to a threshold tumor heterogeneity score.

In some embodiments, the method further comprises treating the subject with a second anti-cancer therapy selected at least in part based on the knowledge of the driver mutation if the tumor heterogeneity score is greater than the threshold tumor heterogeneity score. In some embodiments, the method further comprises determining a clonality metric for the driver mutation, and treating the subject with the first anti-cancer therapy if the tumor heterogeneity score is less than or equal to the threshold tumor heterogeneity score and the clonality metric is greater than or equal to a threshold clonality metric for the first anti-cancer therapy. In some embodiments, the method further comprises treating the subject with the second anti-cancer therapy if the tumor heterogeneity score is greater than the threshold tumor heterogeneity score and the clonality metric is less than the threshold clonality metric for the first anti-cancer therapy. In some embodiments, if the tumor heterogeneity score is greater than the threshold tumor heterogeneity score, the subject is identified for tumor monitoring.

Disclosed herein are methods of predicting a duration of therapeutic response for a subject having a cancer, comprising: acquiring knowledge of a driver mutation in a sample obtained from the subject; determining a tumor heterogeneity score for the sample obtained from the subject; and comparing the tumor heterogeneity score for the sample to a threshold tumor heterogeneity score, wherein if the tumor heterogeneity score is less than or equal to the threshold tumor heterogeneity score, the subject is predicted to have a longer duration of therapeutic response to an anti-cancer therapy selected at least in part based on the knowledge of the driver mutation; and wherein if the tumor heterogeneity score is greater than the threshold tumor heterogeneity score, the subject is predicted to have a shorter therapeutic response to the anti-cancer therapy selected at least in part based on the knowledge of the driver mutation.

Disclosed herein are methods of predicting survival of a subject having a cancer comprising: acquiring knowledge of a driver mutation in a sample obtained from the subject; determining a tumor heterogeneity score for the sample obtained from the subject; and comparing the tumor heterogeneity score for the sample to a threshold tumor heterogeneity score, wherein if the tumor heterogeneity score is less than or equal to the threshold tumor heterogeneity score, the subject is predicted to have increased survival if treated with an anti-cancer therapy selected at least in part based on the knowledge of the driver mutation; and wherein if the tumor heterogeneity score is greater than the threshold tumor heterogeneity, the subject is predicted to have reduced survival if treated with the anti-cancer therapy selected at least in part based on the knowledge of the driver mutation.

Disclosed herein are methods of monitoring, evaluating, or screening a subject having a cancer, comprising acquiring knowledge of a driver mutation in a sample obtained from the subject; determining a tumor heterogeneity score for the sample; and comparing the tumor heterogeneity score to a threshold tumor heterogeneity score, wherein if the tumor heterogeneity score is less than or equal to the threshold tumor heterogeneity score, the subject is predicted to have increased survival when treated with an anti-cancer therapy selected at least in part based on the knowledge of the driver mutation, and wherein if the tumor heterogeneity score is greater than the threshold tumor heterogeneity score, the subject is predicted to have reduced survival when treated with the anti-cancer therapy selected at least in part based on the knowledge of the driver mutation.

In some embodiments of any of the methods disclosed herein, if the tumor heterogeneity score is greater than the threshold tumor heterogeneity score, the subject is identified for tumor monitoring. In some embodiments, the increased survival is increased overall survival (OS), increased progression free survival (PFS), increased disease-free survival (DFS), increased objective response rate (ORR), increased time to tumor progression (TTP), increased time to treatment failure (TTF), increased durable complete response (DCR), or increased time to next treatment (TTNT). In some embodiments, the increased survival is increased overall survival (OS). In some embodiments, the increased survival is increased progression free survival (PFS). In some embodiments, the determination of a tumor heterogeneity score is based on sequence read data derived from the sample obtained from the subject. In some embodiments, the sequence read data is obtained by performing next-generation sequencing (NGS), massively parallel sequencing (MPS), whole genome sequencing (WGS), whole exome sequencing (WES), targeted sequencing, direct sequencing, or Sanger sequencing. In some embodiments, the sequence read is obtained by performing next-generation sequencing (NGS). In some embodiments, the sample comprises cells and/or nucleic acids from the cancer. In some embodiments, the sample obtained from the subject comprises a single biopsy sample. In some embodiments, the sample obtained from the subject comprises multiple biopsy samples. In some embodiments, the sample comprises a tissue biopsy sample, a liquid biopsy sample, or a normal control. In some embodiments, the sample comprises a tissue biopsy sample, and the tissue biopsy sample comprises a tumor biopsy, a tumor specimen, or a circulating tumor cell. In some embodiments, the sample comprises a liquid biopsy sample, and the liquid biopsy sample comprises blood, serum, plasma, cerebrospinal fluid, sputum, stool, urine, or saliva. In some embodiments, the cancer is non-small cell lung cancer (NSCLC), prostate cancer, ovarian cancer, breast cancer, melanoma, colorectal cancer, a cholangiocarcinoma, or prostate cancer. In some embodiments, the sample comprises a liquid biopsy sample, and sequence read data for the subject is obtained by sequencing circulating tumor DNA (ctDNA) in the liquid biopsy sample.

In some embodiments of any of the methods disclosed herein, the cancer is bladder cancer, brain cancer, breast cancer, cervical cancer, colorectal cancer, dermatofibrosarcoma protuberans, an endocrine/neuroendocrine tumor, esophageal cancer, head and neck cancer, a gastrointestinal stromal tumor, a giant cell tumor, kidney cancer, leukemia, liver and bile duct cancer, lung cancer, lymphoma, a malignant mesothelioma, a microsatellite instability-high or mismatch repair-deficient solid tumor, multiple myeloma, a myelodysplastic/myeloproliferative disorder, a neuroblastoma, an ovarian epithelial/fallopian tube/primary peritoneal cancer, pancreatic cancer, a plexiform neurofibroma, prostate cancer, skin cancer, a soft tissue sarcoma, a solid tumor having a high tumor mutational burden (TMB-H), a solid tumor comprising a neurotrophic tyrosine receptor kinase (NTRK) gene fusion, stomach (gastric) cancer, a systemic mastocytosis, a thyroid cancer, or any combination thereof.

In some embodiments, the cancer is acute lymphoblastic leukemia (Philadelphia chromosome positive), acute lymphoblastic leukemia (precursor B-cell), acute myeloid leukemia (FLT3+), acute myeloid leukemia (with IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), colorectal cancer (MSI-H/dMMR), cryopyrin-associated periodic syndromes, cutaneous T-cell lymphoma, dermatofibrosarcoma protuberans, diffuse large B-cell lymphoma, fallopian tube cancer, follicular B-cell non-Hodgkin lymphoma, follicular lymphoma, gastric cancer, gastric cancer (HER2+), gastroesophageal junction (GEJ) adenocarcinoma, gastrointestinal stromal tumors, gastrointestinal stromal tumor (KIT+), giant cell tumor of the bone, glioblastoma, granulomatosis with polyangiitis, head and neck squamous cell carcinoma, hepatocellular carcinoma, Hodgkin lymphoma, juvenile idiopathic arthritis, lupus erythematosus, mantle cell lymphoma, medullary thyroid cancer, melanoma, melanoma (with BRAF V600 mutation), melanoma (with BRAF V600E or V600K mutation), merkel cell carcinoma, multicentric Castleman's disease, multiple hematologic malignancies including Philadelphia chromosome-positive ALL and CML, multiple myeloma, myelofibrosis, non-Hodgkin's lymphoma, nonresectable subependymal giant cell astrocytoma associated with tuberous sclerosis, non-small cell lung cancer, non-small cell lung cancer (ALK+), non-small cell lung cancer (PD-L1+), non-small cell lung cancer (with ALK fusion or ROS1 gene alteration), non-small cell lung cancer (with BRAF V600E mutation), non-small cell lung cancer (with EGFR exon 19 deletions or exon 21 substitution (L858R) mutations), non-small cell lung cancer (with EGFR T790M mutation), ovarian cancer, ovarian cancer (with BRCA mutation), pancreatic cancer, pancreatic, gastrointestinal, or lung origin neuroendocrine tumor, pediatric neuroblastoma, peripheral T-cell lymphoma, peritoneal cancer, prostate cancer, renal cell carcinoma, rheumatoid arthritis, small lymphocytic lymphoma, soft tissue sarcoma, solid tumors (MSI-H/dMMR), squamous cell cancer of the head and neck, squamous non-small cell lung cancer, thyroid cancer, thyroid carcinoma, urothelial cancer, urothelial carcinoma, or Waldenstrom's macroglobulinemia. In some embodiments, the cancer is a solid tumor.

In some embodiments of any of the methods disclosed herein, the cancer comprises a driver mutation or variant in an ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene, or any combination thereof.

In some embodiments, of any of the methods disclosed herein, the cancer comprises a driver mutation or variant in the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB gene, or any combination thereof.

In some embodiments of any of the methods disclosed herein, the cancer comprises a driver mutation in the ALK, ATM, BARD1, BRAF, BRCA1, BRCA2, BRIP1, CDK12, CHEK1, CHEK2, EGFR, ERBB2 (HER2), FANCL, FGFR2, KRAS, MET, NRAS, NTRK1, NTRK2, NTRK3, PIK3CA, PALB2, RAD51B, RAD51C, RAD51D, RAD54L, OR RET gene.

In some embodiments, the cancer comprises an epidermal growth factor receptor (EGFR) driver mutation. In some embodiments, the EGFR driver mutation comprises an EGFR exon 21 L858R mutation, an EGFR exon 19 deletion, an EGFR T790M mutation, or an EGFR amplification.

In some embodiments, the cancer comprises an ALK driver mutation. In some embodiments, the ALK driver mutation comprises an ALK rearrangement.

In some embodiments, the cancer comprises a BRAF driver mutation. In some embodiments, the BRAF driver mutation comprises a BRAF V600E mutation or a BRAF V600K mutation.

In some embodiments, the cancer comprises a MET driver mutation. In some embodiments, the MET driver mutation comprises a MET single nucleotide variant or a MET indel that leads to MET exon 14 skipping.

In some embodiments, the cancer comprises an ERBB2 (HER2) driver mutation. In some embodiments, the ERBB2 (HER2) driver mutation comprises an ERBB2 (HER2) amplification.

In some embodiments, the cancer comprises a PIK3CA driver mutation. In some embodiments, the PIK3CA driver mutation comprises a PIK3CA C420R mutation, a PIK3CA E542K mutation, a PIK3CA E545A mutation, a PIK3CA E545D mutation, a PIK3CA E545G mutation, a PIK3CA E545K mutation, a PIK3CA Q546E mutation, a PIK3CA Q546R mutation, a PIK3CA H1047L mutation, a PIK3CA H1047R mutation, or a PIK3CA H1047Y mutation.

In some embodiments, the cancer comprises a KRAS driver mutation. In some embodiments, the KRAS driver mutation comprises a KRAS mutation in codon 12, codon 13, exon 2, exon 3, or exon 4.

In some embodiments, the cancer comprises a NRAS driver mutation. In some embodiments, the NRAS driver mutation comprises a mutation in exon 2, exon 3, or exon 4.

In some embodiments, the cancer comprises a BRCA1 driver mutation. In some embodiments, the cancer comprises a BRCA2 driver mutation. In some embodiments, the cancer comprises an ATM driver mutation.

In some embodiments, the cancer comprises an FGFR2 driver mutation. In some embodiments, the FGFR2 driver mutation comprises an FGFR2 fusion or an FGFR2 rearrangement.

In some embodiments, the cancer comprises a driver mutation in a homologous recombination repair (HRR) gene. In some embodiments, the driver mutation in the homologous recombination repair (HRR) gene comprises a BRCA1, BRCA2, ATM, BARD1, BRIP1, CDK12, CHEK1, CHEK2, FANCL, PALB2, RAD51B, RAD51C, RAD51D, or RAD54L mutation.

In some embodiments, the cancer comprises an NTRK1 driver mutation. In some embodiments, the NTRK1 driver mutation comprises an NTRK1 fusion.

In some embodiments, the cancer comprises an NTRK2 driver mutation. In some embodiments, the NTRK2 driver mutation comprises an NTRK2 fusion.

In some embodiments, the cancer comprises an NTRK3 driver mutation. In some embodiments, the NTRK3 driver mutation comprises an NTRK3 fusion.

In some embodiments, the cancer comprises a RET driver mutation. In some embodiments, the RET driver mutation comprises a RET fusion.

In some embodiments of any of the methods disclosed herein, the threshold tumor heterogeneity score ranges in value from 0.1 to 20. In some embodiments, the threshold tumor heterogeneity score ranges in value from 0.5 to 2.0. In some embodiments, the threshold clonality metric ranges in value from 0.1 to 0.9. In some embodiments, the threshold clonality metric ranges in value from 0.4 to 0.6.

In some embodiments of any of the methods disclosed herein, the first anti-cancer agent, second anti-cancer agent, first anti-cancer treatment, second anti-cancer treatment, candidate treatment, first anti-cancer therapy, second anti-cancer therapy, or selected anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aligopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubega), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some embodiments of any of the methods disclosed herein, the first anti-cancer agent, second anti-cancer agent, first anti-cancer treatment, second anti-cancer treatment, candidate treatment, first anti-cancer therapy, second anti-cancer therapy, or selected anti-cancer therapy comprises Gilotrif® (afatinib), Iressa® (gefitinib), Tagrisso® (osimertinib), Tarceva® (erlotinib), Alecensa® (alectinib), Alunbrig® (brigatinib), Xalkori® (crizotinib), Zykadia® (ceritinib), Tafinlar® (dabrafenib), Mekinist® (trametinib), Tabrecta® (capmatinib), Tecentriq® (atezolizumab), Cotellic® (cobimetinib), Zelboraf® (vemurafenib), Herceptin® (trastuzumab), Kadcyla® (ado-trastuzumabemtansine), Perjeta® (pertuzumab), Piqray® (alpelisib), Erbitux® (cetuximab), Vectibix® (panitumumab), Lynparza® (olaparib), Rubraca® (rucaparib), Pemazyre® (pemigatinib), Truseltiq™ (infigratinib), Keytruda® (pembrolizumab), Vitrakvi® (larotrectinib), Mekinist® (Trametinib), or Retevmo (selpercatinib).

In some embodiments, the first anti-cancer agent, second anti-cancer agent, first anti-cancer treatment, second anti-cancer treatment, candidate treatment, first anti-cancer therapy, second anti-cancer therapy, or selected anti-cancer therapy comprises an EGFR tyrosine kinase inhibitor. In some embodiments, the first anti-cancer agent, second anti-cancer agent, first anti-cancer treatment, second anti-cancer treatment, candidate treatment, first anti-cancer therapy, second anti-cancer therapy, or selected anti-cancer therapy comprises Osimertinib.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of a process flowchart for determining a tumor heterogeneity score that functions as a biomarker for predicting the estimated duration of a patient's response to a therapy for treating a disease.

FIG. 2 provides a non-limiting example of a process flowchart for determining one or more tumor heterogeneity score (THS) thresholds that divide a patient cohort into two or more response duration groups based on their tumor heterogeneity scores.

FIG. 3 provides another non-limiting example of a process flowchart for selecting a treatment and treating a patient based on comparison of a tumor heterogeneity score to one or more predetermined THS thresholds.

FIG. 4 provides a non-limiting example of a process flowchart for determining whether or not to recommend serial monitoring of a patient receiving a selected disease therapy.

FIG. 5 depicts an exemplary computing device or system in accordance with one embodiment of the present disclosure.

FIG. 6 depicts an exemplary computer system or computer network, in accordance with some instances of the systems described herein.

FIG. 7 provides a schematic illustration of the tumor heterogeneity score for non-small cell lung cancer (NSCLC) patients who have an EGFR driver alteration (e.g., an EGFR L858R mutation or EGFR exon 19 deletion) that is the prime target for an EGFR targeted therapy, in accordance with some instances of the methods and systems described herein.

FIG. 8 provides a study design/cohort diagram for a cohort of non-small cell lung cancer (NSCLC) patients used in evaluating tumor heterogeneity score as a biomarker for the duration of a patient's response to therapy, in accordance with some instances of the methods and systems described herein.

FIG. 9 provides a plot of progression free survival (PFS) probability versus time from start of 1^{st} line Osimertinib treatment for the NSCLC patient cohort stratified by tumor heterogeneity tertile, in accordance with some instances of the methods and systems described herein.

FIG. 10 provides a plot of the results for a Cox proportional hazards regression model from start of 1^{st} line Osimertinib treatment for the NSCLC patient cohort stratified by tumor heterogeneity tertile, in accordance with some instances of the methods and systems described herein.

FIG. 11 provides a plot of progression free survival (PFS) probability versus time from start of 1^{st} line Osimertinib treatment for the NSCLC patient cohort stratified by a binary tumor heterogeneity score (THS) threshold of 1, in accordance with some instances of the methods and systems described herein.

FIG. 12 provides a plot of the results for a Cox proportional hazards regression model from start of 1^{st} line Osimertinib treatment for the NSCLC patient cohort stratified by a binary tumor heterogeneity score (THS) threshold of 1, in accordance with some instances of the methods and systems described herein.

FIG. 13 provides a plot of the individual components of the tumor heterogeneity (TH) score along with information about the binary score category and driver EGFR alteration's cancer cell fraction (CCF), in accordance with some instances of the methods and systems described herein.

FIG. 14 provides a plot of progression free survival (PFS) probability versus time from start of 1^{st} line Osimertinib treatment for the NSCLC patient cohort stratified by a binary tumor heterogeneity score (THS) threshold of 1 and the underlying EGFR driver alteration's clonality, in accordance with some instances of the methods and systems described herein.

FIG. 15 provides a plot of the results for a Cox proportional hazards regression model from start of 1^{st} line Osimertinib treatment for the NSCLC patient cohort stratified by a binary tumor heterogeneity score (THS) threshold of 1 and the underlying EGFR driver alteration's clonality, in accordance with some instances of the methods and systems described herein.

### DETAILED DESCRIPTION

Methods, devices, and systems for determining a tumor heterogeneity score based on genomic data for an individual patient are described, where the score is predictive of the estimated duration of the individual patient's response to a selected treatment for a given disease, e.g., a cancer. Also described are methods for selecting a treatment and for treating a patient based on the tumor heterogeneity score. The disclosed methods have the potential to improve healthcare outcomes for patients (e.g., cancer patients) by providing healthcare providers with a better decision-making tool for guiding 1L and 2L treatment selection and for making recommendations for post-treatment patient monitoring. The disclosed methods for determining a tumor heterogeneity score may also for provide patients with a better understanding of their own prognosis.

In some instances, for example, methods for predicting an estimated duration of a patient's response to a therapy for treating a disease are described that comprise: receiving genomic data for the patient, wherein the genomic data for the patient comprises sequence read data indicative of a presence or absence of one or more short variants in a sample derived from the patient; determining a plurality of cancer cell fraction (CCF) measures by calculating a CCF measure for each of a plurality of short variants present in the genomic data for the patient; determining a tumor heterogeneity score (THS) based on the plurality of CCF measures; comparing the tumor heterogeneity score for the patient to one or more predetermined THS thresholds; and predicting, using the one or more processors, the estimated duration of the patient's response to the therapy for treating the disease based on the comparison.

In some instances, the method further comprises: determining a CCF measure for a driver mutation of the disease present in the genomic data of the patient; and predicting, using a combination of the tumor heterogeneity score and the CCF measure for the driver mutation, the estimated duration of the patient's response to the therapy for treating the disease. In some instances, the prediction is further based on a comparison of the tumor heterogeneity score to a predetermined THS threshold and comparison of the CCF measure for the driver mutation to a predetermined CCF threshold.

In some instances, methods for identifying an individual having a cancer for treatment with a candidate therapy are described, where the methods comprise determining a tumor heterogeneity score for a sample obtained from the individual, wherein if the tumor heterogeneity score is less than or equal to a threshold tumor heterogeneity score for the candidate therapy the individual is identified for treatment with the candidate therapy.

In some instances, methods of selecting a treatment for an individual having a cancer are described, the methods comprising determining a tumor heterogeneity score for a sample obtained from the individual, wherein a tumor heterogeneity score that is less than or equal to a threshold tumor heterogeneity score for a candidate treatment the individual is identified as one who may benefit from treatment with the candidate treatment.

In some instances, the methods further comprise determining a clonality metric for a cancer driver mutation present in the sample obtained from the individual, wherein if the tumor heterogeneity score is less than or equal to a threshold tumor heterogeneity score for the candidate therapy and the clonality metric is greater than or equal to a threshold clonality metric for the candidate treatment, the individual is identified for treatment with the candidate therapy or as one who may benefit from treatment with the candidate treatment.

Also described are methods of identifying one or more treatment options for an individual having a cancer, the methods comprising: a) determining a tumor heterogeneity score for a sample obtained from the individual; and b) generating a report comprising one or more treatment options identified for the individual, wherein a tumor heterogeneity score that is less than or equal to a corresponding threshold tumor heterogeneity score for each of one or more candidate treatment options identifies the individual as one who may benefit from that treatment option.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

The section headings used herein are for organizational purposes only and are not to be construed as limiting the subject matter described.

### Methods for Determining a Tumor Heterogeneity Score

As noted above, healthcare providers often face a multitude of first line (1L) treatment (e.g., the initial therapy selected for treating a patient following a diagnosis of disease, e.g., cancer) and second line (2L) treatment (e.g., a treatment selected after progression/recurrence has occurred following 1L treatment, or a treatment selected following recurrence within 12 months of neoadjuvant/adjuvant treatment) options, and would thus benefit from having access to a prognostic biomarker that helps guide the decision-making for patient treatment selection and follow-up care. For example, patients who may be predicted to become resistant to a given targeted therapy within nine months of initial treatment might be good candidates for serial monitoring (e.g., through the measurement of circulating tumor DNA (ctDNA) dynamics) in order to facilitate expedient 2L treatment decisions. In some instances, tumor monitoring may comprise, for example, follow-up tumor sequencing within 3, 6, 9, or 12 months, or having a CT scan of the subject performed within 3, 6, 9, or 12 months. In contrast, patients who are predicted to have long term, durable benefit to a particular therapy may require less monitoring, and may give patients a greater sense of ownership over their own treatment strategy.

The potential advantages of the methods disclosed herein (and systems configured to perform those methods) include, but are not limited to:
(i) determination of a prognostic tumor heterogeneity score based on single region bulk tumor sequencing (e.g., from a single tissue biopsy sample). Due to the risks involved in performing surgeries and lack of access to extensive tissue biopsies, multi-region sequencing is not clinically feasible. The ability to determine a prognostic tumor heterogeneity score based on single region sequencing data is a result that would have been viewed as unpredictable by one of skill in the art based on previously published studies.
(ii) determination of a prognostic tumor heterogeneity score based on a targeted exome sequencing panel that is used as part of routine clinical care (e.g., as opposed to the whole exome sequencing panels often used in research studies). Again, this is a result that would have been viewed as unpredictable by one of skill in the art based on previously published studies.
(iii) determination of a prognostic tumor heterogeneity score based on, e.g., the calculation of a cancer cell fraction (CCF) for every short variant detected in a patient specimen, and generation of a tumor heterogeneity score based on central tendency (e.g., a mean, median, or mode) and dispersion measurements (e.g., a standard deviation, inter-quartile range, or quartile coefficient of dispersion (QCD)) for the distribution of CCF values calculated for all detected short variants.
(iv) determination of a prognostic tumor heterogeneity score without a requirement for modeling the noise distribution in allele frequencies of the detected short variants used to determine tumor heterogeneity, as has been described in previously published studies (see, e.g., Jamal-Hanjani, et al. ibid.). This makes the disclosed tumor heterogeneity score straight forward to calculate and more easily interpretable in terms of understanding the degree of oncogene addiction (i.e., the process in which cancers comprising genetic, epigenetic, and/or chromosomal irregularities become dependent on one or more genes for maintenance and survival) of the tumor.

As noted above, the disclosed methods have the potential to improve healthcare outcomes for patients (e.g., cancer patients) by providing healthcare providers with a better decision-making tool for guiding 1L and 2L treatment selection and for making recommendations for post-treatment patient monitoring, and may also for provide patients with a better understanding of their own prognosis.

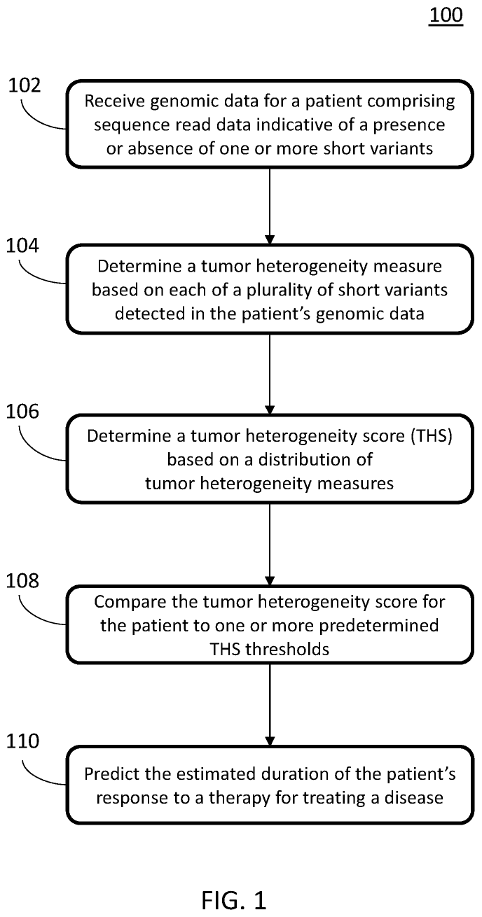

FIG. 1 provides a non-limiting example of a flowchart for a process 100 of determining a tumor heterogeneity score that functions as a biomarker for predicting the estimated duration of a patient's response to a therapy for treating a disease. Process 100 can be performed, for example, using one or more electronic devices implementing a software platform. In some examples, process 100 is performed using a client-server system, and the blocks of process 100 are divided up in any manner between the server and a client device. In other examples, the blocks of process 100 are divided up between the server and multiple client devices. Thus, while portions of process 100 are described herein as being performed by particular devices of a client-server system, it will be appreciated that process 100 is not so limited. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

At step 102 in FIG. 1, genomic data for a patient diagnosed with a disease (e.g., cancer) is received (e.g., by one or more processors of a system configured to perform process 100), where the genomic data comprises sequence read data (derived from, e.g., targeted exome sequencing) that is indicative of a presence or absence of one or more short variants (SVs) in a patient sample.

In some instances, the genomic data may also comprise sequence read data that is indicative of the presence or absence of other genomic features in addition to short variants, such as copy number alterations, rearrangements, chromosomal aneuploidy, whole genome doubling, Catalogue Of Somatic Mutations In Cancer (COSMIC) mutational signatures, or any combination thereof.

In some instances, the genomic data comprising sequence read data may be derived from single region sequencing (e.g., sequencing of a single tissue biopsy sample collected from the patient tumor). In some instances, the genomic data comprising sequence read data may be derived from multi-region sequencing (e.g., sequencing of multiple tissue biopsy samples collected from the patient tumor). In some instances, the genomic data comprising sequence read data may be derived from single cell sequencing data as opposed to bulk tumor sequencing. In some instances, the genomic data comprising sequence read data may be derived from sequencing the circulating tumor DNA in a liquid biopsy sample.

In some instances, the genomic data comprising sequence read data may be derived from, e.g., whole genome or whole exome sequencing, as opposed to targeted exome sequencing to increase the number of genomic features (e.g., the number of short variants) detected.

At step 104 in FIG. 1, a measure of tumor heterogeneity may be calculated based on the presence of each of a plurality of short variants (e.g., short insertions or deletions (indels), single nucleotide polymorphisms (SNPs), single nucleotide variants (SNVs), and the like) detected in the patient's genomic data (including, in some instances, noncoding and synonymous short variants).

In some instances, for example, a cancer cell fraction (CCF) can be indicative of the proportion of the cancer cells present in the tumor that contain a given short variant. In some instances, a CCF value may be calculated based on one or more parameters (e.g., including allele frequency, the number of mutant copies of the gene in which the short variant occurs, the total number of copies of the gene in which the short variant occurs, and a tumor purity measure). In some instances, a CCF value may be calculated for one or more short variants the are present in genomic data (e.g., sequence read data) derived from a sample. In some instances, CCF values may be calculated for each of a plurality of short variants detected in the genomic data (e.g., sequence read data) derived from a sample: In some instances, a CCF value may be calculated for one or more short variants, or for each short variant in a plurality of short variants, detected in a patient's genomic data irrespective of short variant functional status (e.g., known, likely, unknown, or variant of unknown significance (VUS)) or short variant coding type (e.g., synonymous, nonsynonymous, or non-coding). In one non-limiting example, CCF may be calculated according to the following formula:

$$C C F = \frac{f}{m ρ} ( ρ {N}_{T} + 2 ( 1 - ρ ) )$$

where f is the allele frequency of the short variant, m is the number of mutant copies of the gene (i.e., the number of copies of the gene in which the short variant occurs), ρ is tumor purity, and N_{T} is the total number of copies of the gene. In some instances, these values are derived from a computational pipeline for analyzing sequence read data and detecting short variants as well as estimating the somatic/germline origins of the detected short variants.

In some instances, a cancer cell fraction (CCF) may be calculated for specimens and short variants that pass a set of quality control criteria. as illustrated in the non-limiting example shown in Table 1. In some instances, patient samples for which only one of its detected short variants is evaluable for CCF calculation are excluded from further analysis.

TABLE 1

| Exemplary QC criteria for short variants to be evaluated for CCF calculation. | |
|---|---|
| Attribute | Conditions |
| Computational tumor purity | Not missing and >= 20% |
| Total Copies of the SV | Not missing and # 0 |
| Altered Copies of the SV | Not missing |
| Mutant allele frequency | Not missing and <= 1 |
| SGZ call | Contains only “somatic”, “subclonal somatic”, “ambiguous”, “nocall” |
| SGZ consensus call | Does not contain “germline” |
| QC flags | Contains only “.” (no QC flags) |
| DNA QC status | Contains only “Pass” |
| Model switch | Is “FALSE” (referring to a model used in a copy number calling pipeline) |
| Loh QC status | Contains only “Pass” |

In some instances, the measure of tumor heterogeneity may be calculated based on the presence of one or more short variants (e.g., short insertions or deletions (indels), single nucleotide polymorphisms (SNPs), single nucleotide variants (SNVs), and the like) detected in sequence read data derived from a patient sample. In some instances, the measure of tumor heterogeneity may be calculated based on the presence of a plurality of short variants detected in sequence read data derived from a patient sample. In some instances, the measure of tumor heterogeneity may be calculated based on each of a plurality of short variants detected in sequence read data derived from a patient sample. In some instances, the sequence read data may be generated using, e.g., whole genome or whole exome sequencing, as opposed to targeted exome sequencing to increase the number of genomic features (e.g., the number of short variants) detected and incorporate them into the tumor heterogeneity calculation.

In some instances, the measure of tumor heterogeneity may be calculated based on at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 500, at least 1000, at least 2000, at least 3000, at least 4000, at least 5000, at least 6000, at least 7000, at least 8000, at least 9000, at least 10,000, or more than 10,000 short variants detected in the genomic data for a patient.

In some instances, the measure of tumor heterogeneity may be calculated based on other genomic features in addition to short variants, such as copy number alterations, rearrangements, chromosomal aneuploidy, whole genome doubling, Catalogue Of Somatic Mutations In Cancer (COSMIC) mutational signatures, or any combination thereof.

At step 106 in FIG. 1, a tumor heterogeneity score may be determined based on a distribution of the tumor heterogeneity measures determined at step 104. For example, in some instances, the tumor heterogeneity score may be based on central tendency (e.g., a mean, median, mode, geometric mean, or harmonic mean) and dispersion measurements (e.g., a standard deviation, inter-quartile range, or quartile coefficient of dispersion (QCD)) for the distribution of tumor heterogeneity measures (e.g., CCF values) calculated for all short variants detected in the patient sample. In some instances, a tumor heterogeneity score may be determined based on the median of the distance of CCF values of all detected short variants from the CCF value of the primary oncogene identified in the specimen.

In some instances, the tumor heterogeneity score may be determined as the ratio of the median value of the distribution of CCF measures calculated for all short variants detected in the patient sample to the quartile coefficient of dispersion (QCD) of the distribution of CCF measures. In some instances, the tumor heterogeneity score may be viewed as an indirect measure of the oncogene addiction of the tumor.

In some instances, the tumor heterogeneity score may comprise a continuous-valued (e.g., floating point) number and may be reported as such. In some instances, a continuous-valued tumor heterogeneity score may be converted to a binary valued score (e.g., a high score or low score) and reported as such by comparison to a predetermined tumor heterogeneity score (THS) threshold. In some instances, a continuous-valued tumor heterogeneity score may be converted to a categorized score (e.g., a high score, medium score, or low score) and reported as such by comparison to first and second predetermined tumor heterogeneity score (THS) thresholds.

In some instances, the tumor heterogeneity score may incorporate a characterization metric, e.g., a distance of all short variant CCF values present in the genomic data for the patient from that of a targetable driver mutation present in the genomic data for the patient.

In some instances, the tumor heterogeneity score may be continuous-valued and may range in value from 0.1 to 20. In some instances, the tumor heterogeneity score may be at least 0.1, at least 0.2, at least 0.3, at least 0.4, at least 0.5, at least 0.6, at least 0.7, at least 0.8, at least 0.9, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20. In some instances, the tumor heterogeneity score may be at most 20, at most 19, at most 18, at most 17, at most 16, at most 15, at most 14, at most 13, at most 12, at most 11, at most 10, at most 9, at most 8, at most 7, at most 6, at most 5, at most 4, at most 3, at most 2, at most 1, at most 0.9, at most 0.8, at most 0.7, at most 0.6, at most 0.5, at most 0.4, at most 0.3, at most 0.2, or at most 0.1. Any of the lower and upper values described in this paragraph may be combined to form a range included within the present disclosure, for example, in some instances the tumor heterogeneity score may range in value from about 0.2 to about 17. Those of skill in the art will recognize that in some instances, the tumor heterogeneity score may have any value within this range, e.g., about 14.3.

In some instances, the tumor heterogeneity score may be normalized so that it lies within a defined range of values, e.g., such that it ranges in value from 0.05 to 1.0. In some instances, the tumor heterogeneity score (e.g., the normalized tumor heterogeneity score) may be at least 0.05, at least 0.1, at least 0.2, at least 0.3, at least 0.4, at least 0.5, at least 0.6, at least 0.7, at least 0.8, at least 0.9, at least 0.95, or 1.0. In some instances, the tumor heterogeneity score (e.g., the normalized tumor heterogeneity score) may be at most 1, at most 0.95, at most 0.9, at most 0.8, at most 0.7, at most 0.6, at most 0.5, at most 0.4, at most 0.3, at most 0.2, at most 0.1, or at most 0.05. Any of the lower and upper values described in this paragraph may be combined to form a range included within the present disclosure, for example, in some instances the tumor heterogeneity score (e.g., the normalized tumor heterogeneity score) may range in value from about 0.2 to about 0.8. Those of skill in the art will recognize that in some instances, the tumor heterogeneity score (e.g., the normalized tumor heterogeneity score) may have any value within this range, e.g., about 0.64.

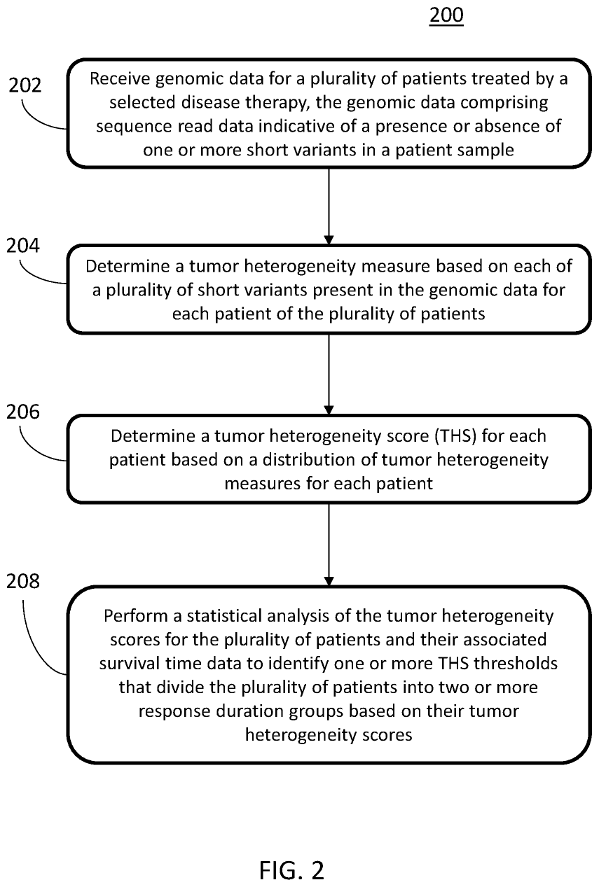

At step 108 in FIG. 1, the tumor heterogeneity score for the patient is compared to one or more predetermined THS thresholds that stratify patient cohorts for a selected therapy into different response duration (patient survival) categories. Methods for determining the one or more predetermined THS thresholds will be described in more detail with respect to FIG. 2 below.

In some instances, for example, the one or more predetermined THS thresholds may be based on stratification of a cohort of patients treated with the therapy into two or more groups of patients, each group having a different estimated duration of patient response to the therapy. The one or more predetermined THS thresholds may thus be determined for a given therapy based on one or more datasets comprising patient survival data for a cohort of patients treated with the given therapy, and may vary for different therapies. In some instances, the one or more predetermined THS thresholds may comprise a first predetermined THS threshold, where a tumor heterogeneity score for the patient that is greater than or equal to the first predetermined THS threshold is indicative of a shorter estimated duration of the patient's response to the therapy for treating the disease. In some instances, the one or more predetermined THS thresholds may comprise a second predetermined THS threshold, where a tumor heterogeneity score for the patient that is less than the second predetermined THS threshold is indicative of a longer estimated duration of the patient's response to the therapy for treating the disease. In some instances, the second predetermined THS threshold is the same as the first predetermined THS threshold. In some instances, the second predetermined THS threshold is different from the first predetermined THS threshold.

In some instances, the one or more predefined THS thresholds (based on a statistical analysis used to stratify a cohort of patients diagnosed with a disease and treated with a selected treatment into two or more patient groups, each having a different estimate of patient response) may each independently have values ranging from 0.1 to 20. In some instances, the one or more predefined THS thresholds may each independently be at least 0.1, at least 0.2, at least 0.3, at least 0.4, at least 0.5, at least 0.6, at least 0.7, at least 0.8, at least 0.9, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20. In some instances, the one or more predefined THS thresholds may each independently be at most 20, at most 19, at most 18, at most 17, at most 16, at most 15, at most 14, at most 13, at most 12, at most 11, at most 10, at most 9, at most 8, at most 7, at most 6, at most 5, at most 4, at most 3, at most 2, at most 1, at most 0.9, at most 0.8, at most 0.7, at most 0.6, at most 0.5, at most 0.4, at most 0.3, at most 0.2, or at most 0.1. Any of the lower and upper values described in this paragraph may be combined to form a range included within the present disclosure, for example, in some instances one or more of the predefined THS thresholds may range in value from about 0.8 to about 3. Those of skill in the art will recognize that in some instances, the predefined THS thresholds may have any value (and different values) within this range, e.g., 0.9 for a first threshold and 2.5 for a second threshold. In some instances, the one or more predefined THS thresholds may be evaluated on a cancer type and/or selected therapy (e.g., targeted therapy) basis.

At step 110 in FIG. 1, the comparison of the tumor heterogeneity score for the patient to the one or more predetermined THS thresholds is used to estimate the likely or estimated duration of the patient's response to a selected therapy for treating a disease, e.g., cancer. In some instances, the tumor heterogeneity score may be compared to two or more different sets of predetermined THS thresholds (each determined for a different selected therapy), so that the patient's tumor heterogeneity score may be used to guide the selection of a first line and/or a second line therapy for treatment of the disease for which the patient has been diagnosed.

In some instances, the ability to generate sequence read data and calculate a tumor heterogeneity measure using only a single biopsy sample may confer advantages in terms of minimizing the invasiveness of the sample collection procedure, thereby reducing the number of visits required, reducing the level of patient discomfort involved, reducing the risk involved with undergoing multiple biopsy procedures, providing more accurate tumor purity determinations for the sample, and/or reducing the overall cost of the sample collection and sample sequencing.

In some instances, the predictive value of the tumor heterogeneity score may be enhanced when used in combination with spatial and temporal information derived from histopathological images, radiological images, magnetic resonance images, ultrasound images, X-ray images, bone scans, CT scans, PET scans, or any combination thereof. In some instances, tumor heterogeneity may be evaluated by computational pathology algorithms, e.g., by processing pathology slide images using a machine learning approach. In some instance, the tumor heterogeneity score may be used in combination with a measurement of circulating tumor fraction at baseline, which is another prognostic biomarker for response. In some instances, the tumor heterogeneity score may be used for assessing circulating tumor fraction at baseline via a liquid biopsy sample (e.g., a blood sample) and/or for selecting patients for serial monitoring.

In some instances, the method for predicting an estimated duration of a patient's response to a therapy for a disease may further comprise determining a CCF measure for a driver mutation of the disease present in the genomic data of the patient; and predicting the estimated duration of the patient's response to the therapy using a combination of the tumor heterogeneity score and the CCF measure for the driver mutation. For example, in some instances, the prediction may be based on a comparison of the tumor heterogeneity score to one or more predetermined THS thresholds and comparison of the CCF measure for the driver mutation to one or more predetermined CCF thresholds.

In some instances, for example, the predetermined CCF threshold may be based on stratification of a cohort of patients treated with the therapy into two groups of patients, each group having a different estimated duration of patient response to the therapy. One or more predetermined CCF thresholds may be determined for a given driver mutation and/or given therapy based on one or more datasets comprising patient survival data for a cohort of patients treated with the given therapy, and may vary for different therapies. In some instances, a CCF measure for the patient that is less than the predetermined CCF threshold is indicative of a shorter estimated duration of the patient's response to the therapy. In some instances, a CCF measure for the patient that is greater than or equal to the predetermined CCF threshold is indicative of a longer estimated duration of the patient's response to the therapy.

In some instances, the CCF measure may be continuous-valued and may range in value from 0.1 to 1.0. In some instances, the CCF measure may be at least 0.1, at least 0.2, at least 0.3, at least 0.4, at least 0.5, at least 0.6, at least 0.7, at least 0.8, at least 0.9, or at least 1.0. In some instances, the CCF measure may be at most 1.0, at most 0.9, at most 0.8, at most 0.7, at most 0.6, at most 0.5, at most 0.4, at most 0.3, at most 0.2, or at most 0.1. Any of the lower and upper values described in this paragraph may be combined to form a range included within the present disclosure, for example, in some instances the CCF measure may range in value from about 0.2 to about 0.8. Those of skill in the art will recognize that in some instances, the CCF measure may have any value within this range, e.g., about 0.45. In some instances, an experimentally-determined CCF measure may exceed a value of 1.0 due to errors in tumor purity estimation and copy number modeling. Improvements in the latter may lead to more accurate determinations of CCF measures.

Similarly, in some instances, the one or more predefined CCF thresholds (based on a statistical analysis used to stratify a cohort of patients diagnosed with a disease and treated with a selected treatment into two or more patient groups, each having a different estimate of patient response) may each independently have values ranging from 0.1 to 1.0. In some instances, the one or more predefined CCF thresholds may each independently be at least 0.1, at least 0.2, at least 0.3, at least 0.4, at least 0.5, at least 0.6, at least 0.7, at least 0.8, at least 0.9, or at least 1.0. In some instances, the one or more predefined CCF thresholds may each independently be at most 1.0, at most 0.9, at most 0.8, at most 0.7, at most 0.6, at most 0.5, at most 0.4, at most 0.3, at most 0.2, or at most 0.1. Any of the lower and upper values described in this paragraph may be combined to form a range included within the present disclosure, for example, in some instances one or more of the predefined CCF thresholds may range in value from about 0.4 to about 0.6. Those of skill in the art will recognize that in some instances, the predefined CCF thresholds may have any value (and different values) within this range, e.g., 0.3 for a first threshold and 0.55 for a second threshold.

### Methods for Determining One or More Thresholds to Stratify Patient Cohorts Based on a Tumor Heterogeneity Score

FIG. 2 provides a non-limiting example of a flowchart for a process 200 for determining one or more THS thresholds that divide a patient cohort into two or more response duration groups based on their tumor heterogeneity scores and associated patient survival data for a selected treatment.

At step 202 in FIG. 2, genomic data for a plurality of patients diagnosed with a disease (e.g., a cancer patient cohort) and treated with a selected disease therapy is received (e.g., by one or more processors of a system configured to perform process 200), where the genomic data for each patient comprises sequence read data (derived, e.g., from targeted exome sequencing) that is indicative of a presence or absence of one or more short variants (SVs) in a sample from the patient.

In some instances, the genomic data for each patient may also comprise sequence read data that is indicative of the presence or absence of other genomic features in addition to short variants, such as copy number alterations, rearrangements, chromosomal aneuploidy, whole genome doubling, Catalogue Of Somatic Mutations In Cancer (COSMIC) mutational signatures, or any combination thereof.

In some instances, the genomic data for each patient comprising sequence read data may be derived from single region sequencing (e.g., sequencing of a single tissue biopsy sample collected from a patient tumor). In some instances, the genomic data comprising sequence read data may be derived from multi-region sequencing (e.g., sequencing of multiple tissue biopsy samples collected from a patient tumor). In some instances, the genomic data comprising sequence read data may be derived from single cell sequencing data as opposed to bulk tumor sequencing. In some instances, the genomic data comprising sequence read data may be derived from sequencing the circulating tumor DNA in a liquid biopsy sample collected from a patient.

In some instances, the genomic data for each patient comprising sequence read data may be derived from, e.g., whole genome or whole exome sequencing, as opposed to targeted exome sequencing to increase the number of genomic features (e.g., the number of short variants) detected.

At step 204 in FIG. 2, a measure of tumor heterogeneity may be calculated for each patient of the plurality, where the tumor heterogeneity measure is based on the presence of each of a plurality of short variants (e.g., short insertions or deletions (indels), single nucleotide polymorphisms (SNPs), single nucleotide variants (SNVs), and the like) detected in a patient's genomic data.

In some instances, for example, the measure of tumor heterogeneity for each patient may comprise a calculation of cancer cell fraction (CCF) for every short variant detected in the patient's genomic data, as described above for FIG. 1.

In some instances, the measure of tumor heterogeneity for each patient of the plurality may be calculated based on the presence of each of a plurality of short variants (e.g., short insertions or deletions (indels), single nucleotide polymorphisms (SNPs), single nucleotide variants (SNVs), and the like) detected in sequence read data derived from, e.g., whole genome or whole exome sequencing, as opposed to targeted exome sequencing to increase the number of genomic features (e.g., the number of short variants) detected and incorporate them into the tumor heterogeneity calculation.

In some instances, the measure of tumor heterogeneity may be calculated for each patient of the plurality of patients based on other genomic features in addition to short variants, such as copy number alterations, rearrangements, chromosomal aneuploidy, whole genome doubling, Catalogue Of Somatic Mutations In Cancer (COSMIC) mutational signatures, or any combination thereof.

At step 206 in FIG. 2, a tumor heterogeneity score is determined for each patient of the plurality of patients based on a distribution of tumor heterogeneity measures determined at step 204. For example, in some instances, the tumor heterogeneity score for each patient of the plurality may be based on central tendency (e.g., a mean, median, or mode) and dispersion measurements (e.g., a standard deviation, inter-quartile range, or quartile coefficient of dispersion (QCD)) for the distribution of tumor heterogeneity measures (e.g., CCF values) calculated for all short variants detected in the patient sample.

In some instances, the tumor heterogeneity score for each patient of the plurality of patients may be determined as the ratio of the median value of the distribution of CCF measures calculated for all short variants detected in the patient sample to the quartile coefficient of dispersion (QCD) of the distribution of CCF measures.

In some instances, the tumor heterogeneity score for each patient may incorporate a metric that characterizes a distance of all short variant CCF values present in the genomic data for the patient from that of a targetable driver mutation present in the genomic data for the patient.

At step 208 in FIG. 2, a statistical analysis of the tumor heterogeneity scores for the plurality of patients and their associated survival time data may be performed to identify one or more THS thresholds that divide the plurality of patients into two or more response duration groups based on their tumor heterogeneity scores, and where the tumor heterogeneity score for an individual patient is predictive of the estimated duration of an individual patient's response to the therapy for treating the disease.

In some instances, the associated patient survival time data may comprise, for example, mean overall survival data, median overall survival data, one-year survival data, hazard ratio data, progression free survival data, or any combination thereof. Because the determination of THS thresholds that may be used to stratify the patient cohort is dependent on an analysis of patient survival data, the THS thresholds may vary for different treatments.

In some instances, for example, the statistical analysis may comprise fitting a regression model. In some instances, the statistical analysis may comprise a Cox proportional hazards regression model—a regression model used to investigate the association between patient survival time following initiation of a selected disease treatment (as expressed by a hazard function) and one or more predictor variables—in this case, tumor heterogeneity score (see, e.g., Bradburn, et al. (2003), “Survival Analysis Part II: Multivariate Data Analysis—An Introduction to Concepts and Methods”, British Journal of Cancer 89, 431-436). In a proportional hazards model, a specified increase in a given covariate results in a proportional scaling of the hazard. A univariable Cox proportional hazards regression model may be used to assess the correlation between patient survival time and a single predictor variable. The multivariable Cox proportional hazards regression model extends the survival analysis method to assess simultaneously the effect of several predictor variables (or risk factors) on survival time.

In some instances, a THS threshold that significantly stratifies patient response to a therapy (e.g., to identify patients predicted to have a low hazard ratio with a p value <0.05 from a univariate or multivariate Cox model) may be determined, e.g., by increasing the THS threshold in a stepwise or continuous manner starting from 0.1 and monitoring the hazard ratio and p value at every threshold value until there are a meaningful number of patients in the low and high groups. In some instances, one may determine THS thresholds from a receiver-operating characteristic (ROC) curve used to predict patient response (e.g., categorical RECIST response (a standardized way to measure how cancer patients respond to treatment) or patient survival outcomes; see, e.g., Irwin, et al. (2011), “A Principled Approach to Setting Optimal Diagnostic Thresholds: Where ROC and Indifference Curves Meet”, European Journal of Internal Medicine, 22(3):230-234).

In some instances, a machine learning model may be used to determine a THS threshold and/or to predict the duration of a patient's therapeutic response to an anti-cancer therapy. Machine learning models used for survival analysis, such as random survival forest, xgboost, glmboost, ridge regression, elasticnet, coxboost, random forest minimal depth, etc., may be leveraged to identify more accurate THS thresholds and/or to predict duration of a patient's therapeutic response.

The multivariable Cox proportional hazards regression model is based on the hazard function, h(t), which describes the risk of dying at time t under a specified set of conditions (e.g., following treatment of a given patient cohort by a specified disease therapy), and is given by the equation:

$$h ( t ) = {h}_{o} ( t ) + exp ( {b}_{1} {x}_{1} + {b}_{2} {x}_{2} + … + {b}_{p} {x}_{p} )$$

where t is the survival time, h(t) is the hazard function determined by a set of p covariates (x₁, x₂, . . . . , xₚ), the coefficients (b₁, b₂, . . . , bₚ) describe the relative impact of the corresponding covariates, and hₒ is the baseline hazard. The multivariable Cox model can thus be viewed as a multiple linear regression of the logarithm of h(t) on the variables xᵢ, with the baseline hazard corresponding to an ‘intercept’ term that varies with time. The quantities exp(bᵢ) are called hazard ratios (HR). A value of bᵢ greater than zero (or a hazard ratio of greater than one) indicates that as the value of the corresponding covariate increases, the event hazard increases and thus the length of survival decreases. A value of bᵢ equal to zero (or a hazard ratio equal to one) indicates that the corresponding covariate has no effect on hazard or length of survival. A value of bᵢ less than zero (or a hazard ratio of less than one) indicates that as the value of the corresponding covariate increases, the event hazard decreases and thus the length of survival increases.

In some instances, a Cox proportional hazards regression model may be trained on the patient cohort dataset (e.g., fit to the patient cohort data) to determine the values of the one or more coefficients (b₁, b₂, . . . , bₚ) that provide the most accurate correlation between the set of covariates and patient survival times. For example, in some instances, a stepwise regression procedure (e.g., a bidirectional stepwise regression procedure) may be used to train the Cox proportional hazards regression model. Stepwise regression is a method of fitting regression models in which the choice of predictive variables is carried out in an automated fashion. At each step, a variable is considered for addition to, or subtraction from, the set of predictive variables included in the model based on a specified criterion, e.g., a forward, backward, or combined sequence of F-tests or t-tests. Examples of the approaches used for stepwise regression are:
Forward selection, in which—starting with no candidate variables included in the model—candidate variables are tested for inclusion using a specified model fit criterion, and added to the model if their inclusion gives a statistically significant improvement of the model fit; the process is repeated until there are no remaining candidate variables for which inclusion provides a statistically significant improvement of the model;
Backward elimination, in which—starting with all candidate variables included in the model—deletion of candidate variables is tested using a specified model fit criterion, and the candidate variables whose loss gives the most statistically insignificant deterioration of the model fit are deleted; the process is repeated until no additional variables can be deleted without incurring a statistically significant loss of fit; and
Bidirectional elimination (a combination of forward selection and backward elimination), in which candidate variables are tested at each step using a specified model fit criterion for inclusion or exclusion.

In some instances, other criteria may be used to select a best fit model from a set of candidate models based on different combinations of predictive variables. Examples of such model selection criteria include, but are not limited to, the Akaike information criterion, the Bayesian information criterion, a Calinski Harabasz score, false discovery rate, and the like.

As noted above with regard to FIG. 1, in some instances the method for predicting an estimated duration of a patient's response to a therapy for a disease may further comprise determining a CCF measure for a driver mutation of the disease present in the genomic data of the patient; and predicting the estimated duration of the patient's response to the therapy using a combination of the tumor heterogeneity score and the CCF measure for the driver mutation. For example, in some instances, the prediction may be based on a comparison of the tumor heterogeneity score to one or more predetermined THS thresholds and comparison of the CCF measure for the driver mutation to one or more predetermined CCF thresholds. In these instances, the CCF threshold may be determined using a similar statistical analysis as described above to identify a threshold (or thresholds) that stratifies a cohort of patients treated with the therapy into two (or more) groups of patients, each group having a different estimated duration of patient response to the therapy. In some instances, a CCF measure for the patient that is less than the predetermined CCF threshold is indicative of a shorter estimated duration of the patient's response to the therapy. In some instances, a CCF measure for the patient that is greater than or equal to the predetermined CCF threshold is indicative of a longer estimated duration of the patient's response to the therapy.

### Use of a Tumor Heterogeneity Score as a Biomarker

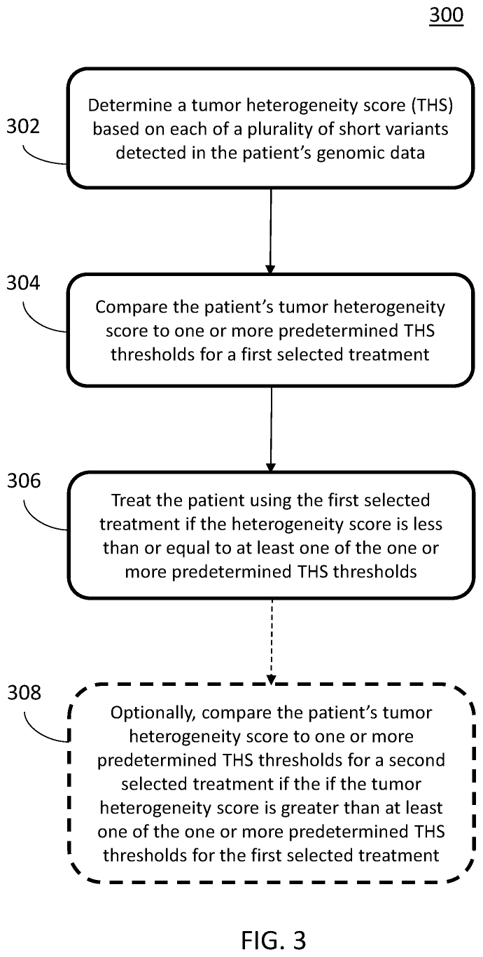

FIG. 3 provides a non-limiting example of a flowchart for a process 300 for selecting a treatment and/or treating a patient diagnosed with a disease (e.g., cancer) based on comparison of a tumor heterogeneity score determined for a sample obtained from the patient to one or more predetermined THS thresholds for each of one or more candidate disease treatments.

At step 302 in FIG. 3, a tumor heterogeneity score for the patient may be calculated based on the presence of each of a plurality of variants identified in the sample obtained from the patient. In some instances, the plurality of variants may comprise short variants (e.g., short insertions or deletions (indels), single nucleotide polymorphisms (SNPs), single nucleotide variants (SNVs), and the like) detected in genomic data for the patient. In some instances, a short variant (or “short variant sequence”) may comprise a variant sequence (e.g., short insertions or deletions (indels), single nucleotide polymorphisms (SNPs), single nucleotide variants (SNVs), and the like) of less than about 50 base pairs in length.

In some instances, the genomic data may comprise sequence read data (derived from, e.g., targeted exome sequencing) indicative of a presence or absence of one or more variants (e.g., short variants). In some instances, the genomic data may also comprise sequence read data that is indicative of the presence or absence of other genomic features in addition to short variants, such as copy number alterations, rearrangements, chromosomal aneuploidy, whole genome doubling, Catalogue Of Somatic Mutations In Cancer (COSMIC) mutational signatures, or any combination thereof.

In some instances, the genomic data comprising sequence read data may be derived from single region sequencing (e.g., sequencing of a single tissue biopsy sample collected from the patient tumor). In some instances, the genomic data comprising sequence read data may be derived from multi-region sequencing (e.g., sequencing of multiple tissue biopsy samples collected from the patient tumor). In some instances, the genomic data comprising sequence read data may be derived from single cell sequencing data as opposed to bulk tumor sequencing. In some instances, the genomic data comprising sequence read data may be derived from sequencing the circulating tumor DNA in a liquid biopsy sample.

In some instances, the genomic data comprising sequence read data may be derived from, e.g., whole genome or whole exome sequencing, as opposed to targeted exome sequencing to increase the number of genomic features (e.g., the number of short variants) detected.

In some instances, a tumor heterogeneity score for the patient may be calculated based on the presence of each of a plurality of short variants (e.g., short insertions or deletions (indels), single nucleotide polymorphisms (SNPs), single nucleotide variants (SNVs), and the like) detected in the patient's genomic data.

In some instances, for example, the tumor heterogeneity score for each patient may comprise a calculation of cancer cell fraction (CCF) for every short variant detected in the patient's genomic data, as described above for FIG. 1. In some instances, determination of the tumor heterogeneity score may further comprise determining a measure of central tendency (e.g., a mean, median, or mode) and a measure of dispersion (e.g., a standard deviation, inter-quartile range, or quartile coefficient of dispersion (QCD)) for the distribution of tumor heterogeneity measures (e.g., CCF values) calculated for all short variants detected in the patient sample. In some instances, for example, the tumor heterogeneity score may be determined as the ratio of the median value of the distribution of CCF measures calculated for all short variants detected in the patient sample to the quartile coefficient of dispersion (QCD) of the distribution of CCF measures.

In some instances, the tumor heterogeneity score may be calculated based on the presence of each of a plurality of short variants (e.g., short insertions or deletions (indels), single nucleotide polymorphisms (SNPs), single nucleotide variants (SNVs), and the like) detected in sequence read data derived from, e.g., whole genome or whole exome sequencing, as opposed to targeted exome sequencing to increase the number of genomic features (e.g., the number of short variants) detected and incorporate them into the tumor heterogeneity calculation.

In some instances, the tumor heterogeneity score may be calculated based on at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 500, at least 1000, at least 2000, at least 3000, at least 4000, at least 5000, at least 6000, at least 7000, at least 8000, at least 9000, at least 10,000,or more than 10,000 short variants detected in the genomic data for a patient.

In some instances, the tumor heterogeneity score may be calculated based on other genomic features in addition to short variants, such as copy number alterations, rearrangements, chromosomal aneuploidy, whole genome doubling, Catalogue Of Somatic Mutations In Cancer (COSMIC) mutational signatures, or any combination thereof.

In some instances, the tumor heterogeneity score may comprise a continuous-valued (e.g., floating point) number and may be reported as such. In some instances, a continuous-valued tumor heterogeneity score may be converted to a binary valued score (e.g., a high-low score) and reported as such by comparison to a predetermined tumor heterogeneity score (THS) threshold. In some instances, a continuous-valued tumor heterogeneity score may be converted to a categorized score (e.g., a high score, medium score, or low score) and reported as such by comparison to first and second predetermined tumor heterogeneity score (THS) thresholds.

In some instances, the tumor heterogeneity score may be continuous-valued and may range in value from 0.1 to 20. In some instances, the tumor heterogeneity score may be at least 0.1, at least 0.2, at least 0.3, at least 0.4, at least 0.5, at least 0.6, at least 0.7, at least 0.8, at least 0.9, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20. In some instances, the tumor heterogeneity score may be at most 20, at most 19, at most 18, at most 17, at most 16, at most 15, at most 14, at most 13, at most 12, at most 11, at most 10, at most 9, at most 8, at most 7, at most 6, at most 5, at most 4, at most 3, at most 2, at most 1, at most 0.9, at most 0.8, at most 0.7, at most 0.6, at most 0.5, at most 0.4, at most 0.3, at most 0.2, or at most 0.1. Any of the lower and upper values described in this paragraph may be combined to form a range included within the present disclosure, for example, in some instances the tumor heterogeneity score may range in value from about 0.2 to about 17. Those of skill in the art will recognize that in some instances, the tumor heterogeneity score may have any value within this range, e.g., about 14.3.

In some instances, the tumor heterogeneity score may be normalized so that it lies within a defined range of values, e.g., such that it ranges in value from 0.05 to 1.0. In some instances, the tumor heterogeneity score (e.g., the normalized tumor heterogeneity score) may be at least 0.05, at least 0.1, at least 0.2, at least 0.3, at least 0.4, at least 0.5, at least 0.6, at least 0.7, at least 0.8, at least 0.9, at least 0.95, or 1.0. In some instances, the tumor heterogeneity score (e.g., the normalized tumor heterogeneity score) may be at most 1, at most 0.95, at most 0.9, at most 0.8, at most 0.7, at most 0.6, at most 0.5, at most 0.4, at most 0.3, at most 0.2, at most 0.1, or at most 0.05. Any of the lower and upper values described in this paragraph may be combined to form a range included within the present disclosure, for example, in some instances the tumor heterogeneity score (e.g., the normalized tumor heterogeneity score) may range in value from about 0.2 to about 0.8. Those of skill in the art will recognize that in some instances, the tumor heterogeneity score (e.g., the normalized tumor heterogeneity score) may have any value within this range, e.g., about 0.64.

Similarly, in some instances, the one or more predefined THS thresholds (based on a statistical analysis used to stratify a cohort of patients diagnosed with a disease and treated with a selected treatment into two or more patient groups, each having a different estimate of patient response) may each independently have values ranging from 0.1 to 20. In some instances, the one or more predefined THS thresholds may each independently be at least 0.1, at least 0.2, at least 0.3, at least 0.4, at least 0.5, at least 0.6, at least 0.7, at least 0.8, at least 0.9, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20. In some instances, the one or more predefined THS thresholds may each independently be at most 20, at most 19, at most 18, at most 17, at most 16, at most 15, at most 14, at most 13, at most 12, at most 11, at most 10, at most 9, at most 8, at most 7, at most 6, at most 5, at most 4, at most 3, at most 2, at most 1, at most 0.9, at most 0.8, at most 0.7, at most 0.6, at most 0.5, at most 0.4, at most 0.3, at most 0.2, or at most 0.1. Any of the lower and upper values described in this paragraph may be combined to form a range included within the present disclosure, for example, in some instances one or more of the predefined THS thresholds may range in value from about 0.8 to about 3. Those of skill in the art will recognize that in some instances, the predefined THS thresholds may have any value (and different values) within this range, e.g., 0.9 for a first threshold and 2.5 for a second threshold. In some instances, the one or more predefined THS thresholds may be evaluated on a cancer type and/or selected therapy (e.g., targeted therapy) basis.

In some instances, the tumor heterogeneity score may incorporate a characterization metric that characterizes, e.g., a distance of all short variant CCF values present in the genomic data for the patient from that of a targetable driver mutation present in the genomic data for the patient.

At step 304 in FIG. 3, the tumor heterogeneity score for the patient is compared to one or more predetermined THS thresholds that stratify patient cohorts for a selected therapy into different response duration (patient survival) categories. For example, if the tumor heterogeneity score is less than or equal to at least one of the predetermined THS thresholds, the patient is predicted to have a longer duration of response to the selected therapy, is predicted to survive for a longer period of time if treated with the selected therapy, or is identified as a patient who would likely benefit from treatment by the selected therapy. Alternatively, if the tumor heterogeneity score is greater than at least one of the predetermined THS thresholds, the patient is predicted to have a shorter duration of response to the selected therapy, is predicted to survive for a shorter period of time, or is identified as a patient who would likely not benefit from treatment by the selected therapy. Methods for determining the one or more predetermined THS thresholds are described in more detail with respect to FIG. 2 above.

At step 306 in FIG. 3, the comparison of the tumor heterogeneity score for the patient to the one or more predetermined THS thresholds performed in step 304 is used to estimate the likely duration of the patient's response to a selected therapy for treating a disease, e.g., cancer, and treat the patient with the selected therapy if the tumor heterogeneity score is less than or equal to at least one of the predetermined THS thresholds. That is, if the tumor heterogeneity score is less than or equal to at least one of the predetermined THS thresholds, the patient is predicted to have a longer duration of response to the selected therapy, is predicted to survive for a longer period of time if treated with the selected therapy, or is identified as a patient who would likely benefit from treatment by the selected therapy. Alternatively, if the tumor heterogeneity score is greater than at least one of the predetermined THS thresholds, the patient is predicted to have a shorter duration of response to the selected therapy, is predicted to survive for a shorter period of time, or is identified as a patient who would likely not benefit from treatment by the selected therapy.

In some instances, the tumor heterogeneity score may be compared to two or more different sets of predetermined THS thresholds (e.g., where each set is determined for a different selected therapy) by repeating step 304, so that the patient's tumor heterogeneity score may be used to guide the selection of a 1L and/or 2L therapy for treatment of the disease for which the patient has been diagnosed.

At step 308 in FIG. 3, the tumor heterogeneity score may optionally be compared to one or more predetermined THS thresholds for a second selected treatment if the if the tumor heterogeneity score is greater than at least one of the one or more predetermined THS thresholds for the first selected treatment. In some instances, for example, a second selected treatment (or treatment option) may comprise a targeted therapy and/or chemotherapy.

As noted above with regard to FIG. 1 and FIG. 2, in some instances the method for predicting an estimated duration of a patient's response to a therapy for a disease and/or selecting a therapy for treatment of the disease may further comprise determining a CCF measure for a driver mutation of the disease present in the genomic data of the patient; and predicting the estimated duration of the patient's response to the therapy using a combination of the tumor heterogeneity score and the CCF measure for the driver mutation. For example, in some instances, the prediction may be based on a comparison of the tumor heterogeneity score to one or more predetermined THS thresholds and comparison of the CCF measure for the driver mutation to one or more predetermined CCF thresholds. In some instances, the use of the tumor heterogeneity score in combination with the CCF measure as a biomarker for patient survival, duration of response, and treatment selection, etc., may be useful in making targeted therapy decisions where a particular driver mutation is present in the tumor. In some instances, the tumor heterogeneity score may have implications in making chemotherapy and/or immunotherapy treatment decisions, in which case a driver alteration may not be relevant.

In some instances, the CCF measure may be continuous-valued and may range in value from 0.1 to 1.0. In some instances, the CCF measure may be at least 0.1, at least 0.2, at least 0.3, at least 0.4, at least 0.5, at least 0.6, at least 0.7, at least 0.8, at least 0.9, or at least 1.0. In some instances, the CCF measure may be at most 1.0, at most 0.9, at most 0.8, at most 0.7, at most 0.6, at most 0.5, at most 0.4, at most 0.3, at most 0.2, or at most 0.1. Any of the lower and upper values described in this paragraph may be combined to form a range included within the present disclosure, for example, in some instances the CCF measure may range in value from about 0.2 to about 0.8. Those of skill in the art will recognize that in some instances, the CCF measure may have any value within this range, e.g., about 0.45. In some instances, an experimentally-determined CCF measure may exceed a value of 1.0 due to errors in tumor purity estimation and copy number modeling. Improvements in the latter may lead to more accurate determinations of CCF measures.

Similarly, in some instances, the one or more predefined CCF thresholds (based on a statistical analysis used to stratify a cohort of patients diagnosed with a disease and treated with a selected treatment into two or more patient groups, each having a different estimate of patient response) may each independently have values ranging from 0.1 to 1.0. In some instances, the one or more predefined CCF thresholds may each independently be at least 0.1, at least 0.2, at least 0.3, at least 0.4, at least 0.5, at least 0.6, at least 0.7, at least 0.8, at least 0.9, or at least 1.0. In some instances, the one or more predefined CCF thresholds may each independently be at most 1.0, at most 0.9, at most 0.8, at most 0.7, at most 0.6, at most 0.5, at most 0.4, at most 0.3, at most 0.2, or at most 0.1. Any of the lower and upper values described in this paragraph may be combined to form a range included within the present disclosure, for example, in some instances one or more of the predefined CCF thresholds may range in value from about 0.4 to about 0.6. Those of skill in the art will recognize that in some instances, the predefined CCF thresholds may have any value (and different values) within this range, e.g., 0.3 for a first threshold and 0.55 for a second threshold.

In some instances, the predictive value of the patient's tumor heterogeneity score may be augmented with spatial and temporal information derived from histopathological images, radiological images, magnetic resonance images, ultrasound images, X-ray images, bone scans, CT scans, PET scans, or any combination thereof.

In some instances, the tumor heterogeneity score, used alone or in combination with a clonality metric (e.g., a CCF calculation) for a driver mutation identified in a sample obtained from an individual, may be used to, e.g., identify an individual diagnosed with cancer for treatment with a selected therapy, select a treatment for an individual based on their genomic data, identify one or more treatment options for the individual, treat the individual with a treatment selected based on the tumor heterogeneity score and/or clonality metric, predict the survival time and/or duration of response for an individual treated with a specific treatment, or any combination thereof.

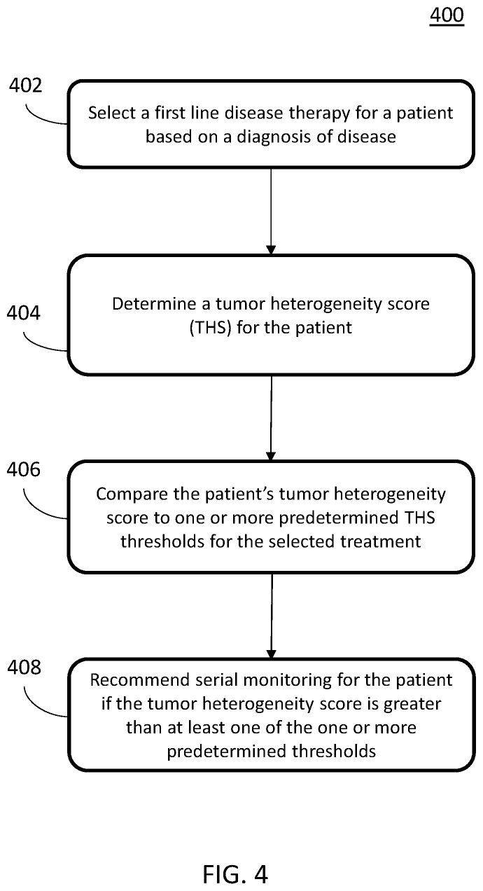

FIG. 4 provides a non-limiting example of a flowchart for a process 400 for determining whether or not to recommend serial monitoring of a patient receiving a selected disease therapy.

At step 402 in FIG. 4, a first line therapy for treating a disease is selected for the patient based on a diagnosis of disease. In some instances, the selection of the first line treatment may be guided solely by clinical indications and patient history. In some instances, the selection of the first line treatment may be guided or augmented by calculating a tumor heterogeneity score for the patient using the processes described in FIG. 1 and FIG. 3.

At step 404 in FIG. 4, a tumor heterogeneity score is calculated for the patient, e.g., according to the processes described in FIG. 1 and FIG. 3.

At step 406 in FIG. 4, the patient's tumor heterogeneity score is compared to one or more predetermined THS thresholds for the selected treatment, where the one or more predetermined THS thresholds stratify patient cohorts for a selected therapy into different response duration (patient survival) categories. For example, if the tumor heterogeneity score is less than or equal to at least one of the predetermined THS thresholds, the patient is predicted to have a longer duration of response to the selected therapy, is predicted to survive for a longer period of time if treated with the selected therapy, or is identified as a patient who would likely benefit from treatment by the selected therapy. Alternatively, if the tumor heterogeneity score is greater than at least one of the predetermined THS thresholds, the patient is predicted to have a shorter duration of response to the selected therapy, is predicted to survive for a shorter period of time, or is identified as a patient who would likely not benefit from treatment by the selected therapy.

At step 408 in FIG. 4, a recommendation for serial monitoring of the patient (e.g., by follow-up sequencing of circulating tumor DNA and re-calculating the patient's tumor heterogeneity score based on the short variants detected) may be made by a healthcare provider if the patient's tumor heterogeneity score is greater than or equal to at least one of the one or more predetermined THS thresholds (e.g., if the tumor heterogeneity score is higher than a predetermined THS threshold and thus indicates a shorter duration of response to the selected treatment by the patient).

In some instances, the method for predicting an estimated duration of a patient's response to a therapy for a disease may further comprise determining a CCF measure for a driver mutation of the disease present in the genomic data of the patient; and predicting the estimated duration of the patient's response to the therapy using a combination of the tumor heterogeneity score and the CCF measure for the driver mutation. For example, in some instances, the prediction may be based on a comparison of the tumor heterogeneity score to one or more predetermined THS thresholds and comparison of the CCF measure for the driver mutation to one or more predetermined CCF thresholds.

In some instances, the tumor heterogeneity score, alone or in combination with a clonality metric for a driver mutation identified in a sample obtained from an individual, may be used by a healthcare provider for predicting a duration of therapeutic response for an individual having a cancer to a selected anti-cancer therapy. In some instances, the tumor heterogeneity score, alone or in combination with a clonality metric for a driver mutation identified in a sample obtained from an individual, may be used by a healthcare provider for predicting survival of an individual having a cancer treated by a selected anti-cancer therapy. In some instances, the tumor heterogeneity score, alone or in combination with a clonality metric for a driver mutation identified in a sample obtained from an individual, may be used by a healthcare provider for identifying an individual having a cancer for treatment with an anti-cancer therapy. In some instances, the tumor heterogeneity score, alone or in combination with a clonality metric for a driver mutation identified in a sample obtained from an individual, may be used by a healthcare provider for selecting a treatment for an individual having a cancer. In some instances, the tumor heterogeneity score, alone or in combination with a clonality metric for a driver mutation identified in a sample obtained from an individual, may be used by a healthcare provider for identifying one or more treatment options for an individual having a cancer. In some instances, the tumor heterogeneity score, alone or in combination with a clonality metric for a driver mutation identified in a sample obtained from an individual, may be used by a healthcare provider for stratifying (or classifying) an individual with a cancer for treatment. In some instances, the tumor heterogeneity score, alone or in combination with a clonality metric for a driver mutation identified in a sample obtained from an individual, may be used by a healthcare provider for making a treatment decision for treating an individual having a cancer. In some instances, the tumor heterogeneity score, alone or in combination with a clonality metric for a driver mutation identified in a sample obtained from an individual, may be used by a healthcare provider for making a decision regarding serial monitoring of the patient. In some instances, the tumor heterogeneity score, alone or in combination with a clonality metric for a driver mutation identified in a sample obtained from an individual, may be used by a healthcare provider for making a decision regarding first line disease therapy for the patient. In some instances, the tumor heterogeneity score, alone or in combination with a clonality metric for a driver mutation identified in a sample obtained from an individual, may be used by a healthcare provider for making a decision regarding second line disease therapy for the patient. In some instances, the second line disease therapy comprises chemotherapy or a targeted immunotherapy.

In some instances, the tumor heterogeneity score may be used, either alone or in combination with a clonality metric for a driver mutation identified in a sample obtained from an individual, to: (i) predict patient survival time, (ii) predict duration of therapeutic response, (iii) identify an individual for treatment with a selected therapy, (iv) identify treatment options for an individual patient, (v) select a therapy for an individual patient, and/or (vi) recommend that an individual patient undergo serial monitoring for any of a variety of cancers. Examples include, but are not limited to, bladder cancer, brain cancer, breast cancer, cervical cancer, colorectal cancer, dermatofibrosarcoma protuberans, an endocrine/neuroendocrine tumor, esophageal cancer, head and neck cancer, a gastrointestinal stromal tumor, a giant cell tumor, kidney cancer, leukemia, liver and bile duct cancer, lung cancer, lymphoma, a malignant mesothelioma, a microsatellite instability-high or mismatch repair-deficient solid tumor, multiple myeloma, a myelodysplastic/myeloproliferative disorder, a neuroblastoma, an ovarian epithelial/fallopian tube/primary peritoneal cancer, pancreatic cancer, a plexiform neurofibroma, prostate cancer, skin cancer, a soft tissue sarcoma, a solid tumor having a high tumor mutational burden (TMB-H), a solid tumor comprising a neurotrophic tyrosine receptor kinase (NTRK) gene fusion, stomach (gastric) cancer, a systemic mastocytosis, a thyroid cancer, or any combination thereof. In some instances, the cancer may be non-small cell lung cancer (NSCLC), prostate cancer, ovarian cancer, breast cancer, melanoma, colorectal cancer, a cholangiocarcinoma, or prostate cancer. In some instances, the cancer may be a solid tumor.

In some instances, the cancer is acute lymphoblastic leukemia (Philadelphia chromosome positive), acute lymphoblastic leukemia (precursor B-cell), acute myeloid leukemia (FLT3+), acute myeloid leukemia (with IDH2 mutation), anaplastic large cell lymphoma, basal cell carcinoma, B-cell chronic lymphocytic leukemia, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), colorectal cancer (MSI-H/dMMR), cryopyrin-associated periodic syndromes, cutaneous T-cell lymphoma, dermatofibrosarcoma protuberans, diffuse large B-cell lymphoma, fallopian tube cancer, follicular B-cell non-Hodgkin lymphoma, follicular lymphoma, gastric cancer, gastric cancer (HER2+), gastroesophageal junction (GEJ) adenocarcinoma, gastrointestinal stromal tumors, gastrointestinal stromal tumor (KIT+), giant cell tumor of the bone, glioblastoma, granulomatosis with polyangiitis, head and neck squamous cell carcinoma, hepatocellular carcinoma, Hodgkin lymphoma, juvenile idiopathic arthritis, lupus erythematosus, mantle cell lymphoma, medullary thyroid cancer, melanoma, melanoma (with BRAF V600 mutation), melanoma (with BRAF V600E or V600K mutation), merkel cell carcinoma, multicentric Castleman's disease, multiple hematologic malignancies including Philadelphia chromosome-positive ALL and CML, multiple myeloma, myelofibrosis, non-Hodgkin's lymphoma, nonresectable subependymal giant cell astrocytoma associated with tuberous sclerosis, non-small cell lung cancer, non-small cell lung cancer (ALK+), non-small cell lung cancer (PD-L1+), non-small cell lung cancer (with ALK fusion or ROS1 gene alteration), non-small cell lung cancer (with BRAF V600E mutation), non-small cell lung cancer (with EGFR exon 19 deletions or exon 21 substitution (L858R) mutations), non-small cell lung cancer (with EGFR T790M mutation), ovarian cancer, ovarian cancer (with BRCA mutation), pancreatic cancer, pancreatic, gastrointestinal, or lung origin neuroendocrine tumor, pediatric neuroblastoma, peripheral T-cell lymphoma, peritoneal cancer, prostate cancer, renal cell carcinoma, rheumatoid arthritis, small lymphocytic lymphoma, soft tissue sarcoma, solid tumors (MSI-H/dMMR), squamous cell cancer of the head and neck, squamous non-small cell lung cancer, thyroid cancer, thyroid carcinoma, urothelial cancer, urothelial carcinoma, or Waldenstrom's macroglobulinemia.

In some instances, the tumor heterogeneity score may be used in combination with a clonality metric determined for a driver mutation identified in a sample from a patient. In some instances, for example, the tumor heterogeneity score and/or a clonality metric may be determined for driver mutations or other alterations (e.g., short variants) in the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene, or any combination thereof. In some instances, the cancer may comprise a driver mutation in the ABL, BCR, BRAF, EGFR, HER-2, or VEGF gene, or any combination thereof. In some instances, the cancer may comprise a driver mutation in the EGFR gene. In some instances, the EGFR driver mutation may comprise an EGFR L858R mutation, an EGFR exon 19 deletion, or an EGFR amplification.

In some instances, the cancer comprises a driver mutation or variant in the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB gene, or any combination thereof.

In some instances, the cancer comprises a driver mutation in the ALK, ATM, BARD1, BRAF, BRCA1, BRCA2, BRIP1, CDK12, CHEK1, CHEK2, EGFR, ERBB2 (HER2), FANCL, FGFR2, KRAS, MET, NRAS, NTRK1, NTRK2, NTRK3, PIK3CA, PALB2, RAD51B, RAD51C, RAD51D, RAD54L, OR RET gene.

In some instances, a first and/or a second line treatment (e.g., an anti-cancer agent, anti-cancer therapy, anti-cancer treatment, or candidate treatment) selected based on a determination of tumor heterogeneity score and/or a clonality metric for a driver mutation present in a sample derived from an individual (e.g., a patient) may comprise abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aligopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubega), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the first anti-cancer agent, second anti-cancer agent, first anti-cancer treatment, second anti-cancer treatment, candidate treatment, first anti-cancer therapy, second anti-cancer therapy, or selected anti-cancer therapy comprises Gilotrif® (afatinib), Iressa® (gefitinib), Tagrisso® (osimertinib), Tarceva® (erlotinib), Alecensa® (alectinib), Alunbrig® (brigatinib), Xalkori® (crizotinib), Zykadia® (ceritinib), Tafinlar® (dabrafenib), Mekinist® (trametinib), Tabrecta® (capmatinib), Tecentriq® (atezolizumab), Cotellic® (cobimetinib), Zelboraf® (vemurafenib), Herceptin® (trastuzumab), Kadcyla® (ado-trastuzumabemtansine), Perjeta® (pertuzumab), Piqray® (alpelisib), Erbitux® (cetuximab), Vectibix® (panitumumab), Lynparza® (olaparib), Rubraca® (rucaparib), Pemazyre® (pemigatinib), Truseltiq™ (infigratinib), Keytruda® (pembrolizumab), Vitrakvi® (larotrectinib), Mekinist® (Trametinib), or Retevmo (selpercatinib).

In some instances, an anti-cancer agent, anti-cancer therapy, anti-cancer treatment, and/or candidate treatment selected based on a determination of tumor heterogeneity score and/or a clonality metric for a driver mutation present in a sample derived from an individual (e.g., a patient) may comprise an EGFR tyrosine kinase inhibitor. In some instances, an anti-cancer agent, anti-cancer therapy, anti-cancer treatment, and/or candidate treatment selected based on a determination of tumor heterogeneity score and/or a clonality metric for a driver mutation present in a sample derived from an individual (e.g., a patient) may comprise Osimertinib.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for determining a tumor heterogeneity score may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for determining a tumor heterogeneity score may be used to select a subject (e.g., a patient) for a clinical trial based on the subject's tumor heterogeneity score. In some instances, patient selection for clinical trials based on, e.g., determination of a tumor heterogeneity score for the subject, may accelerate the development of targeted therapies and improve the healthcare outcomes for treatment decisions.

In some instances, the disclosed methods for determining a tumor heterogeneity score may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy (e.g., immunotherapy), surgery, or any combination thereof.

In some instances, the disclosed methods for determining a tumor heterogeneity score for a subject may be used in selecting a treatment and/or treating a disease (e.g., a cancer) in the subject. For example, in response to determining a tumor heterogeneity score for the subject using any of the methods disclosed herein, an effective amount of an anti-cancer therapy or anti-cancer treatment may be administered to the subject.

In some instances, the disclosed methods for determining a tumor heterogeneity score may be used for monitoring disease progression or recurrence (e.g., cancer or tumor progression or recurrence) in a subject. For example, in some instances, the methods may be used to determine a tumor heterogeneity score in a first sample obtained from the subject at a first time point and used to determine a tumor heterogeneity score in a second sample obtained from the subject at a second time point, where comparison of the first determination of tumor heterogeneity score and the second determination of tumor heterogeneity score allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the determination of tumor heterogeneity score.

In some instances, the value of the tumor heterogeneity score determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for determining a tumor heterogeneity score may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for determining a tumor heterogeneity score as part of a genomic profiling process (or inclusion of the output from the disclosed methods for determining a tumor heterogeneity score as part of the genomic profile of the subject) can improve the validity of, e.g., disease detection calls and treatment decisions, made on the basis of the genomic profile by, for example, independently confirming the selection of a treatment for the patient.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1):35-42; Masuda, et al., (1999) Nucleic Acids Res. 27(22):4436-4443; Specht, et al., (2001) Am J Pathol. 158(2):419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification.

Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome.

For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm).

The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou, D. T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426.

Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLiD, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S.L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1):195-197), the Striped Smith-Waterman algorithm (see, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (1970) “A General Method Applicable to the Search for Similarities in the Amino Acid Sequence of Two Proteins”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized. In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B.L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation-detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9):1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C. A., et al., Genome Res. 2011; 21(6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix-Bioinformatics. 2010 March 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of mutation calling methods is provided in, e.g., International Patent Application Publication No. WO 2020/236941, the entire content of which is incorporated herein by reference.

### Systems

Also disclosed herein are systems designed to implement any of the disclosed methods for determining a tumor heterogeneity score in a sample from a subject. The systems may comprise, e.g., one or more processors, and a memory unit communicatively coupled to the one or more processors and configured to store instructions that, when executed by the one or more processors, cause the system to: receive genomic data for a patient, wherein the genomic data for the patient comprises sequence read data indicative of a presence or absence of one or more short variants in a sample derived from the patient; determine a plurality of cancer cell fraction (CCF) measures by calculating a CCF measure for each of a plurality of short variants present in the genomic data for the patient; determine a tumor heterogeneity score (THS) based on the plurality of CCF measures; compare the tumor heterogeneity score for the patient to one or more predetermined THS thresholds; and predict the estimated duration of the patient's response to a therapy for treating a disease based on the comparison.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLiD) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for determining a tumor heterogeneity score for any of a variety of samples as described herein (e.g., a tissue sample, biopsy sample, hematological sample, or liquid biopsy sample derived from the subject).

In some instances, the plurality of gene loci for which sequencing data is processed to determine a tumor heterogeneity score may comprise at least 1, 2, 3, 4, 5, 6, 7, 8, 9, 10, 20, 30, 40, 50, 60, 70, 80, 90, 100, or more than 100 gene loci.

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of a tumor heterogeneity score is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

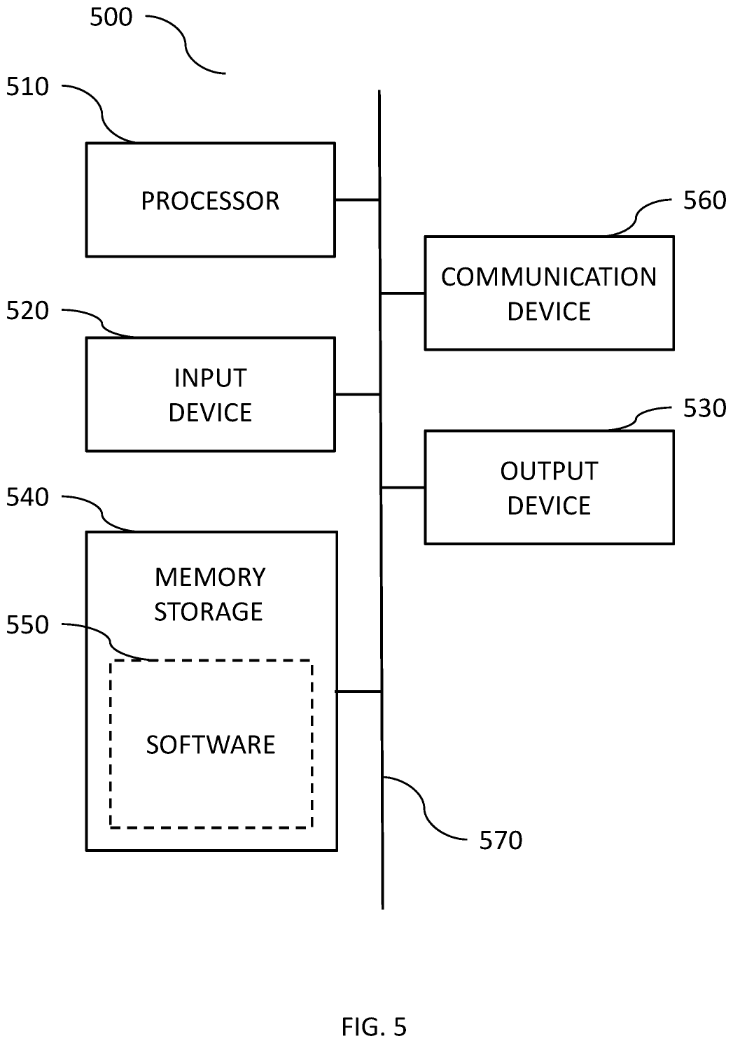

FIG. 5 illustrates an example of a computing device or system in accordance with one embodiment. Device 500 can be a host computer connected to a network. Device 500 can be a client computer or a server. As shown in FIG. 5, device 500 can be any suitable type of microprocessor-based device, such as a personal computer, workstation, server or handheld computing device (portable electronic device) such as a phone or tablet. The device can include, for example, one or more processor(s) 510, input devices 520, output devices 530, memory or storage devices 540, communication devices 560, and nucleic acid sequencers 570. Software 550 residing in memory or storage device 540 may comprise, e.g., an operating system as well as software for executing the methods described herein. Input device 520 and output device 530 can generally correspond to those described herein, and can either be connectable or integrated with the computer.

Input device 520 can be any suitable device that provides input, such as a touch screen, keyboard or keypad, mouse, or voice-recognition device. Output device 530 can be any suitable device that provides output, such as a touch screen, haptics device, or speaker.

Storage 540 can be any suitable device that provides storage (e.g., an electrical, magnetic or optical memory including a RAM (volatile and non-volatile), cache, hard drive, or removable storage disk). Communication device 560 can include any suitable device capable of transmitting and receiving signals over a network, such as a network interface chip or device. The components of the computer can be connected in any suitable manner, such as via a wired media (e.g., a physical system bus 580, Ethernet connection, or any other wire transfer technology) or wirelessly (e.g., Bluetooth®, Wi-Fi®, or any other wireless technology).

Software module 550, which can be stored as executable instructions in storage 540 and executed by processor(s) 510, can include, for example, an operating system and/or the processes that embody the functionality of the methods of the present disclosure (e.g., as embodied in the devices as described herein).

Software module 550 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 540, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 550 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

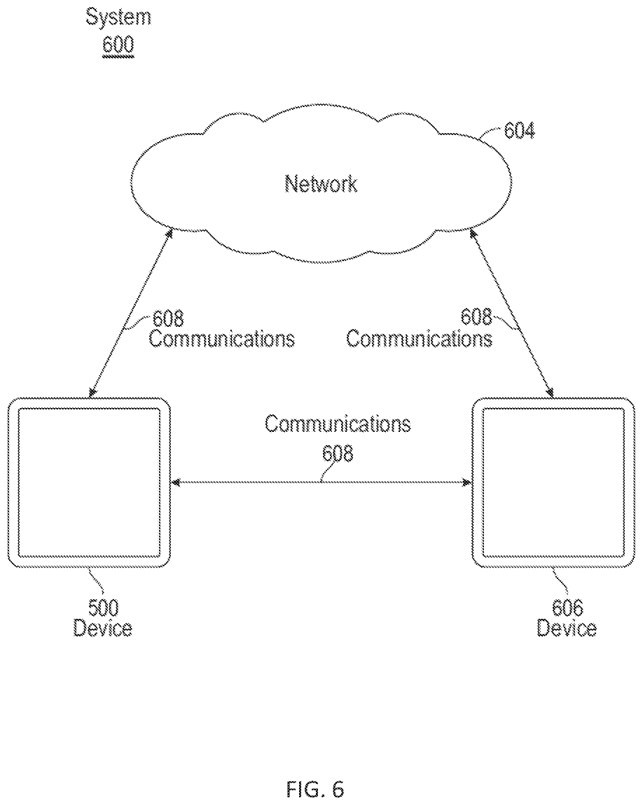

Device 500 may be connected to a network (e.g., network 604, as shown in FIG. 6 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 500 can be implemented using any operating system, e.g., an operating system suitable for operating on the network. Software module 550 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 510.

Device 500 can further include a sequencer 570, which can be any suitable nucleic acid sequencing instrument.

FIG. 6 illustrates an example of a computing system in accordance with one embodiment. In system 600, device 500 (e.g., as described above and illustrated in FIG. 5) is connected to network 604, which is also connected to device 606. In some embodiments, device 606 is a sequencer. Exemplary sequencers can include, without limitation, Roche/454's Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio® RS system.

Devices 500 and 606 may communicate, e.g., using suitable communication interfaces via network 604, such as a Local Area Network (LAN), Virtual Private Network (VPN), or the Internet. In some embodiments, network 604 can be, for example, the Internet, an intranet, a virtual private network, a cloud network, a wired network, or a wireless network. Devices 500 and 606 may communicate, in part or in whole, via wireless or hardwired communications, such as Ethernet, IEEE 802.11b wireless, or the like. Additionally, devices 500 and 606 may communicate, e.g., using suitable communication interfaces, via a second network, such as a mobile/cellular network. Communication between devices 500 and 606 may further include or communicate with various servers such as a mail server, mobile server, media server, telephone server, and the like. In some embodiments, Devices 500 and 606 can communicate directly (instead of, or in addition to, communicating via network 604), e.g., via wireless or hardwired communications, such as Ethernet, IEEE 802.11b wireless, or the like. In some embodiments, devices 500 and 606 communicate via communications 608, which can be a direct connection or can occur via a network (e.g., network 604).

One or all of devices 500 and 606 generally include logic (e.g., http web server logic) or are programmed to format data, accessed from local or remote databases or other sources of data and content, for providing and/or receiving information via network 604 according to various examples described herein.

### EXAMPLES

### Example 1—Visual Depiction of a Tumor Heterogeneity Score for NSCLC Patients Who have an EGFR Driver Alteration

As noted above, healthcare providers would benefit from having access to a prognostic biomarker that helps guide the decision-making for patient treatment selection and follow-up care. Furthermore, clinical solutions such as combination therapies may be used as a multi-pronged approach to target tumors that are genomically heterogeneous. As one non-limiting example of the many potential disease applications for using a tumor heterogeneity score, alone or in combination with a clonality metric for a driver mutation present in a patient sample, as a biomarker for prediction of patient survival, prediction of duration of therapeutic response, or for treatment selection, consider non-small cell lung cancer (NSCLC) patients. Non-small cell lung cancer (NSCLC) patients who progress on Osimertinib (a third-generation epidermal growth factor receptor tyrosine kinase inhibitor (TKI)) due to acquired resistance from a MET amplification (MET is a gene that makes a protein that is involved in sending signals within cells and in cell growth and survival) can be treated with a combination of Osimertinib and MET inhibitor. Healthcare providers are also considering the use of Osimertinib plus carboplatin/etoposide (a chemotherapy used to treat small cell cancers) for those patients with a tumor protein 53 (TP53) mutation; Osimertinib plus a mitogen-activated protein kinase (MEK) inhibitor for those patients with RAS/RAF/MAPK alterations; and Osimertinib plus a fourth-generation EGFR TKI for those patients with EGFR C797 alterations. Thus, a prognostic biomarker based on tumor heterogeneity may be particularly beneficial for guiding treatment decisions in these situations.

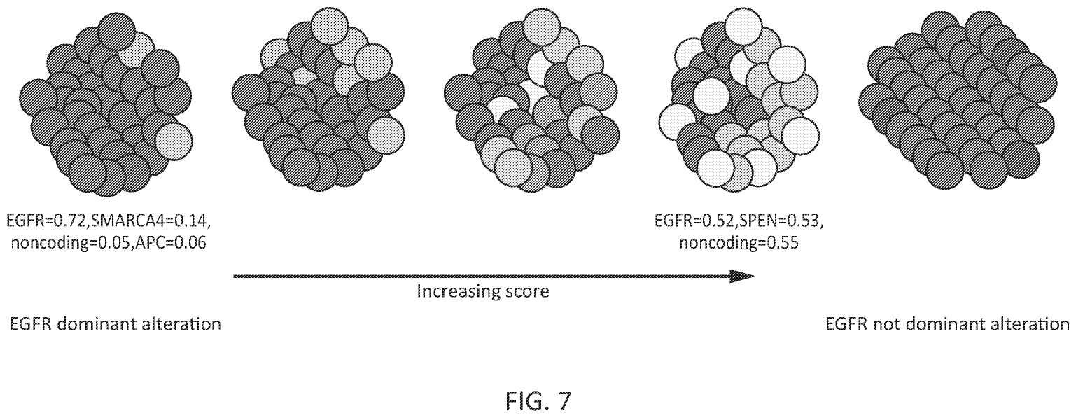

FIG. 7 provides a visual depiction of the tumor heterogeneity score for NSCLC patients who have an EGFR driver alteration (e.g., an EGFR L858R mutation or an EGFR exon 19 deletion), i.e. the alteration that is the prime target for an EGFR targeted therapy. Every circle represents a cell in the tumor, and a bunch of cells represent the tumor mass. Grey circles are cells that have an EGFR driver alteration. Non-grey circles are cells that do not have an EGFR driver alteration. In this illustration, EGFR is assumed to be a clonal alteration (i.e., having a CCF at least 0.5).

A high tumor heterogeneity score indicates that the EGFR oncogene is not the dominant alteration (i.e., the oncogene has competition from other genomic alterations to control the tumor growth), while a low tumor heterogeneity score indicates that the EGFR oncogene is a high CCF outlier and is the tumor driving alteration.

### Example 2—Exploration of the Tumor Heterogeneity Score Through Real World Clinico-Genomic Datasets

This example illustrates the use of tumor heterogeneity score to stratify patient response to first line Osimertinib in EGFR driver alteration-positive NSCLC patients who had their tumor sequenced prior to start of therapy. We show that a tumor heterogeneity score based on the CCF of all eligible short variants detected in a patient specimen is able to stratify response to first line Osimertinib in a real-world NSCLC patient cohort.

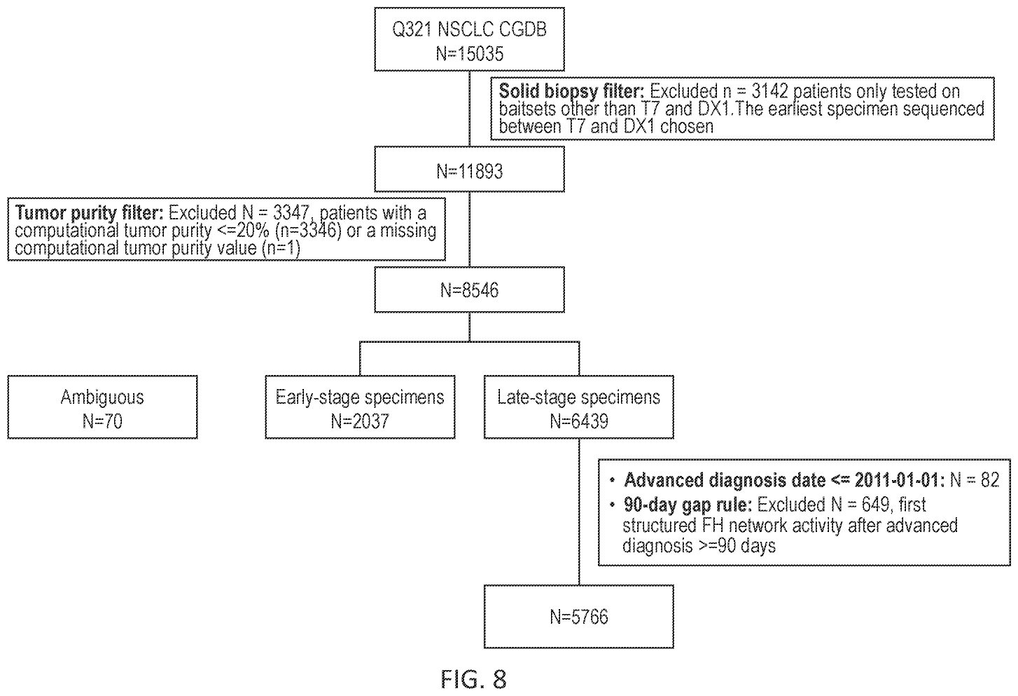

FIG. 8 shows a cohort diagram of the NSCLC cohort. Starting with a patient cohort of 15,035 patients, the data was filtered (for biopsy type and bait set). The data was then filtered to exclude data for patients for whom a computational tumor purity was less than or equal to 20% or for whom a computational tumor purity value was missing. The remaining patient data was divided according to whether the samples were early-stage specimens, late-stage specimens, or of ambiguous origin. For the late-stage specimens, data for patients who were first diagnosed prior to Jan. 1, 2011 and data for patients who failed to meet the 90-day gap rule was also excluded. Finally, data was selected for N=135 1^{st} line Osimertinib-treated, EGFR driver alteration-positive NSCLC patients for whom tumor samples were sequenced at most a year prior to start of therapy.

The demographic characteristics of the NSCLC cohort selected for analysis are summarized in Table 2.

TABLE 2
Demographic Characteristics

| Demographic Characteristics | | Patients (%; N=135) |
|---|---|---|
| Age at initiation of first line therapy, | | 69.0 [63.0-76.0] |
| median (IQR; years) | | |
| Sex | Male | 34.1 |
| | Female | 65.9 |
| Race | Asian | 15.6 |
| | African American | 6.7 |
| | White | 52.6 |
| | Other | 19.3 |
| | Unknown | 5.9 |
| Practice type | Academic | 10.4 |
| | Community | 89.6 |
| Tumor stage at | Stage I | 6.7 |
| initial diagnosis | Stage II | 2.2 |
| | Stage III | 5.2 |
| | Stage IV | 85.2 |
| | Unknown | 0.7 |
| Smoking status | History of smoking | 43.7 |
| | No history of smoking | 56.3 |
| ECOG status at | 0 | 22.2 |
| initiation of therapy | 1 | 31.9 |
| | 2 | 5.2 |
| | 3 | 0.7 |
| | Missing | 40 |

The EGFR alterations detected in the cohort using a sequencing-based variant calling pipeline are summarized in Table 3.

TABLE 3

| EGFR alterations in the cohort. | |
|---|---|
| | Patients (%) |
| EGFR alterations | (N = 135) |
| EGFR L858R | 45.9 |
| EGFR exon 19 deletion | 54.1 |
| EGFR T790M | 1.5 |
| EGFR C797S | 0 |
| EGFR amplifications | 31.8 |

Genomic alterations detected in the cohort that are associated with resistance to Osimertinib are summarized in Table 4.

TABLE 4
Genomic alterations described associated with resistance to Osimertinib

| Osimertinib resistance alterations | Patients (%; N = 135) |
|---|---|
| MET amplifications | 0.7 |
| HER2 amplifications | 1.5 |
| CCND1 amplifications | 3.7 |
| CCND2 amplifications | 0 |
| CCNE1 amplifications | 7.4 |
| CDK4 amplifications | 7.1 |
| CDK6 amplifications | 3.0 |
| ALK fusions | 0 |
| RET fusions | 0 |
| ROS1 fusions | 0 |
| NTRK fusions | 0 |
| FGFR3 fusions | 0 |
| BRAF fusions | 0.7 |
| PIK3CA | 9.6 |
| KRAS | 3.7 |
| TP53 | 65.2 |
| CDKN2A | 37.0 |
| CDKN2B | 32.6 |
| RB1 | 14.8 |

Association of tumor heterogeneity (TH) score with survival outcomes: Amongst the 135 patients, the distribution of the tumor heterogeneity score was as follows: the median score was 1.4 (Inter Quartile Range (IQR): 0.8-2.4; Range: 0.17-15.89). The higher the score, the lower the oncogene (EGFR driver alteration) addiction.

The group of 135 patients were then divided into three tertiles with 45 patients each. Progression free survival (PFS) from start of 1st line Osimertinib was determined as follows:
Index state was the start of 1st line therapy.
Progression date: for patients who received a subsequent line of therapy, the event date was the earliest progression event that occurred more than 14 days after the index date, or date of death, provided that the progression event or date of death occurred before the start date of the subsequent line of therapy plus 14 days. For patients without a subsequent line of therapy, the event date was the earliest progression event that occurred more than 14 days after the index date, or the date of death.
Censor date: patients with a subsequent line of therapy were censored at their last clinic note date if it occurred between the index date and the start date of the subsequent line of therapy plus 14 days. Patients for whom the last clinic note date was not available within this window were censored at their last date of confirmed structured activity (i.e., the last available visit date within the Visit Table) within this time window. Patients without a subsequent line of therapy were censored at their last clinic note date.

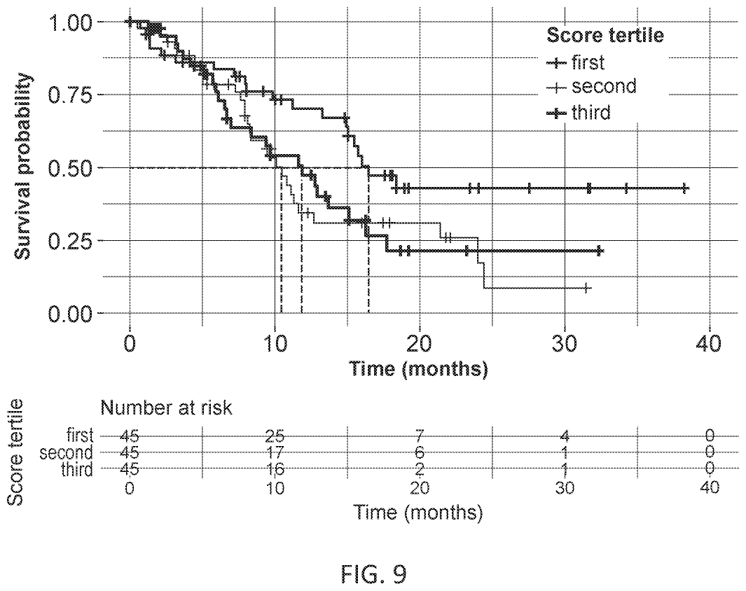

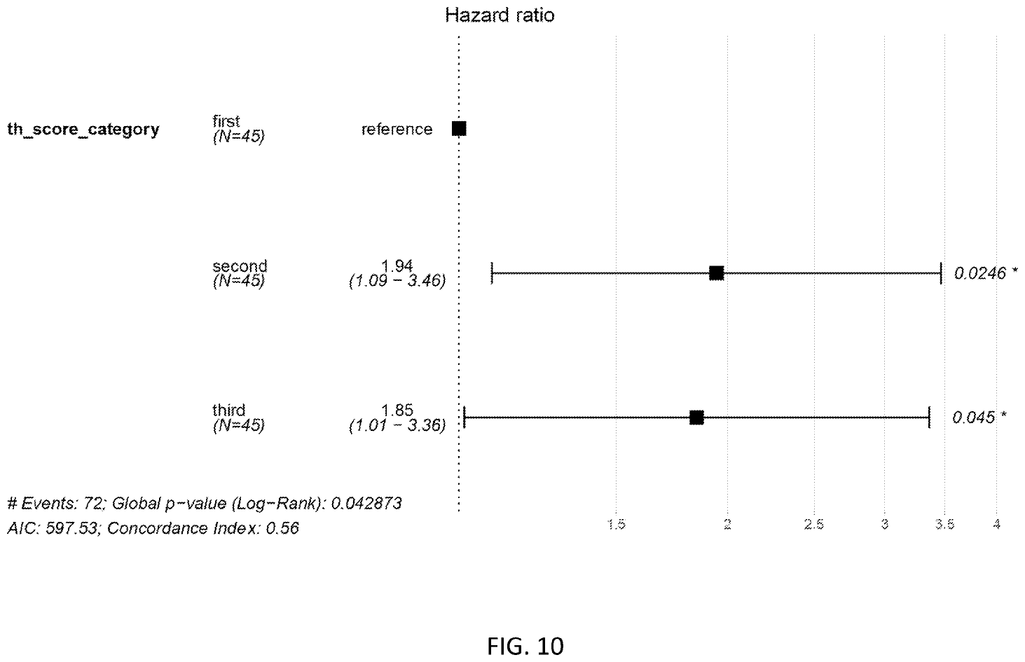

Median progression free survival (mPFS)—from start of 1st line Osimertinib—of the whole cohort was observed to be 12.7 months [range: 10.5-16.0 months]. As shown in FIG. 9 and FIG. 10 below, we observed that the patients in the first tertile (lowest tumor heterogeneity score) had a significantly higher mPFS compared to the other two subgroups.

FIG. 9 provides a plot of progression free survival (PFS) probability versus time from start of 1^{st} line Osimertinib treatment for the NSCLC patient cohort stratified by tumor heterogeneity (TH) score tertile. For the first tertile, the median progression free survival (mPFS) was 16.5 months [15.0-NA], for the second tertile, mPFS was 10.5 months [8.2-21.4], and for the third tertile, mPFS was 11.9 months [7.0-17.7], P=0.04. The lower panel in FIG. 9 provides table of the number of remaining patients at risk as a function of time following initiation of 1^{st} Osimertinib treatment for each of the three tertiles—first (lowest tumor heterogeneity score), second (intermediate tumor heterogeneity score), and third (highest tumor heterogeneity score).

FIG. 10 provides a plot of the results for a Cox proportional hazards regression model from start of 1st line Osimertinib treatment for the NSCLC patient cohort stratified by tumor heterogeneity tertile. The plot shows the hazard ratio for the TH high group versus the TH low group, where the log-rank p-value is the immediately relevant metric. The concordance index is useful when comparing the results to another “TH score”-like metric.

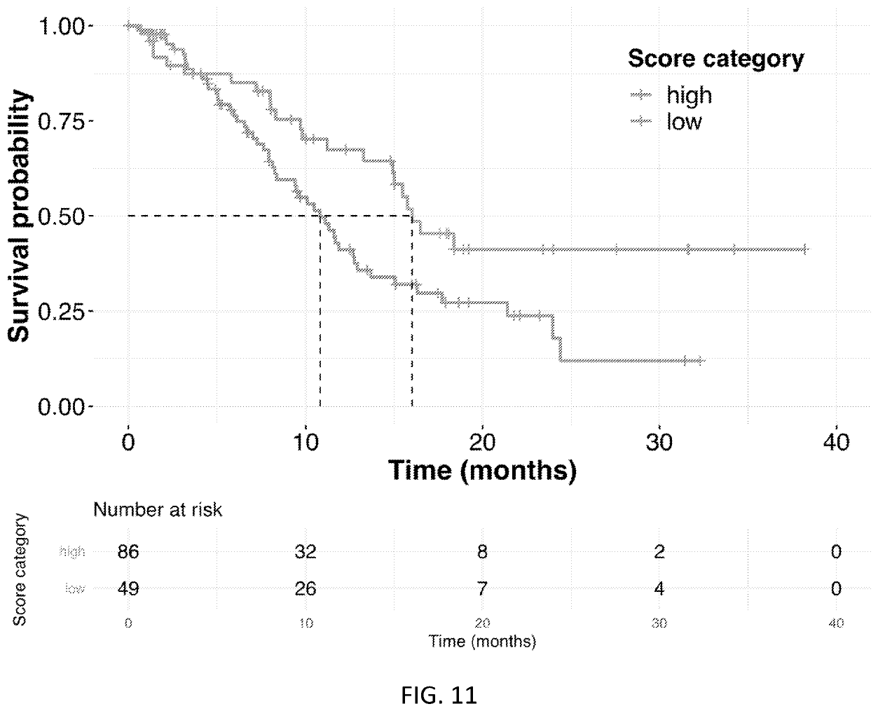

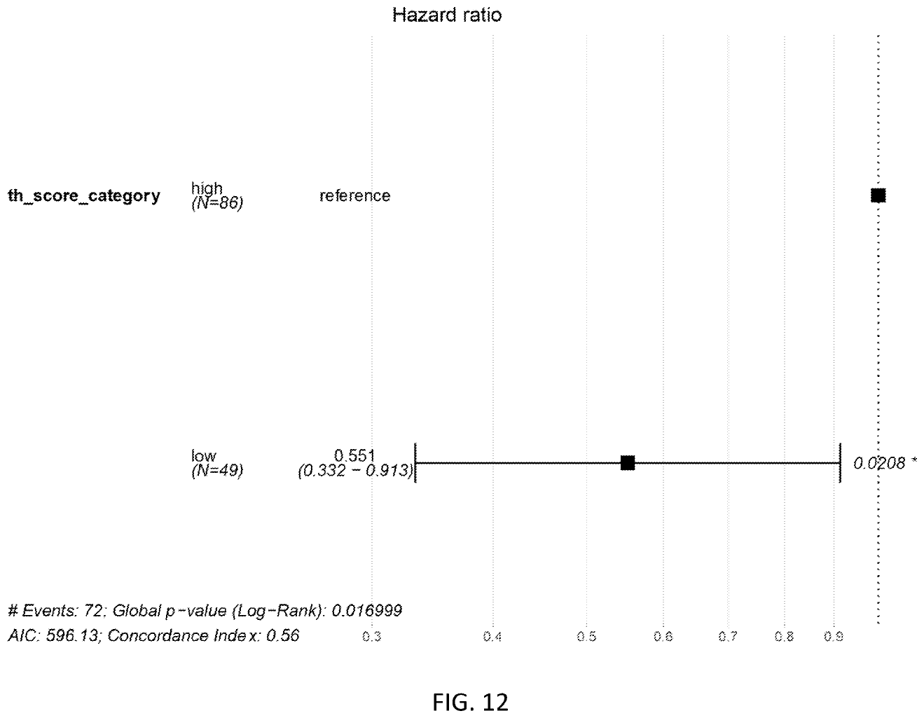

As the second and third tertiles had comparable mPFS in FIG. 9, we divided the cohort into two categories instead of three based on a binary tumor heterogeneity score (THS) threshold of 1. We observed that the patients in the low score group had a significantly higher median PFS compared to the high score group. The mPFS for the low score group was 16.0 months [14.9-NA], and the mPFS for the high score group was 10.8 months [8.4-12.9], P=0.02 (see FIG. 11 and FIG. 12).

FIG. 11 provides a plot of progression free survival (PFS) probability from start of 1^{st} line Osimertinib treatment for the NSCLC cohort stratified by a binary tumor heterogeneity score (THS) threshold of 1. High score group: tumor heterogeneity score >=1. Low score group: tumor heterogeneity score <1. Median PFS for the low score group was 16.0 months [14.9-NA], while the median PFS for the high score group was 10.8 months [8.4-12.9], P=0.02. The lower panel in FIG. 11 provides table of the number of remaining patients at risk as a function of time following initiation of 1st Osimertinib treatment for each of the two binary heterogeneity score groups—the high score group and the low score group.

FIG. 12 provides a plot of the results for a Cox proportional hazards regression model from start of 1^{st} line Osimertinib treatment for the cohort stratified by a binary tumor heterogeneity score (THS) threshold of 1. The plot shows the hazard ratio for the TH high group versus the TH low group. The Akaike information criterion (AIC), log rank p-value and concordance index provide measures of model quality and the significance of the result.

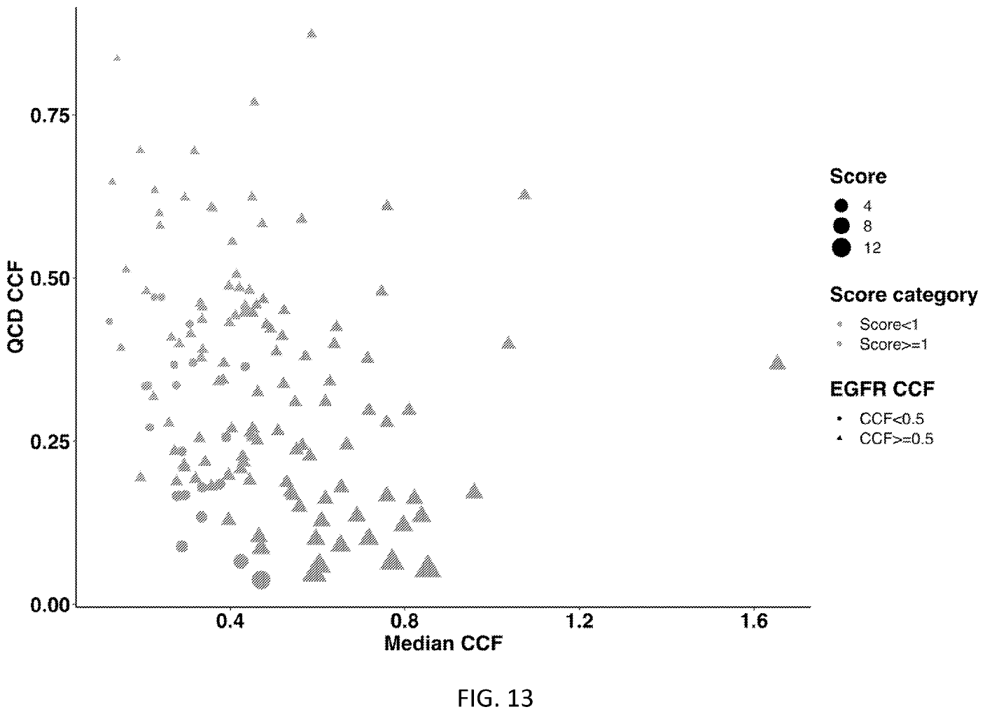

As illustrated schematically FIG. 7, the low score group represents patients where a driver EGFR alteration (e.g., an L858R mutation or an Exon 19 deletion) is the dominating outlier alteration. A more detailed analysis show in FIG. 13 below.

FIG. 13 provides a plot of the individual components of the tumor heterogeneity (TH) score along with information about the binary score category and driver EGFR alteration's cancer cell fraction (CCF). Each dot represents a patient, and patient's median CCF and QCD of CCFs is plotted.

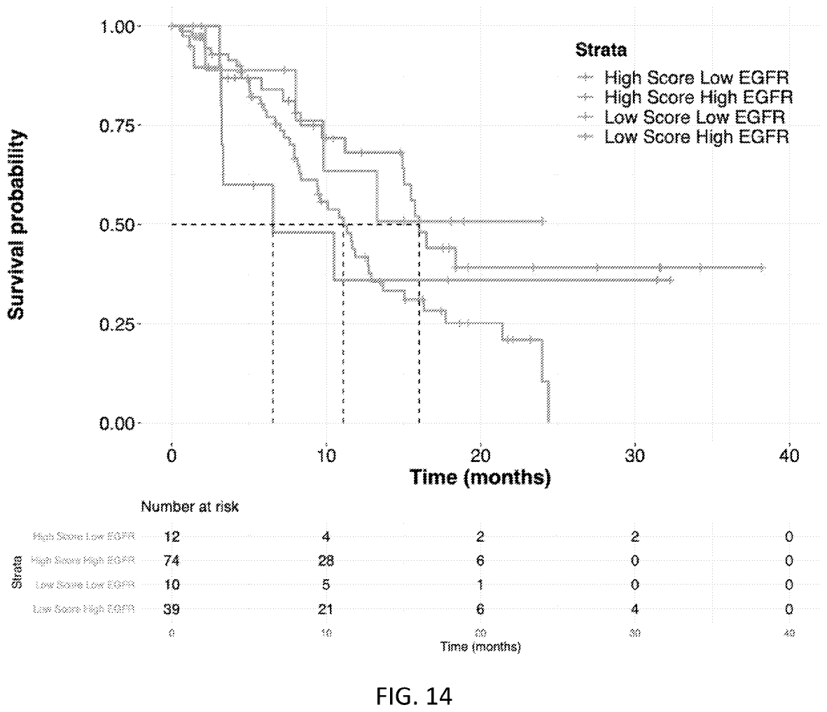

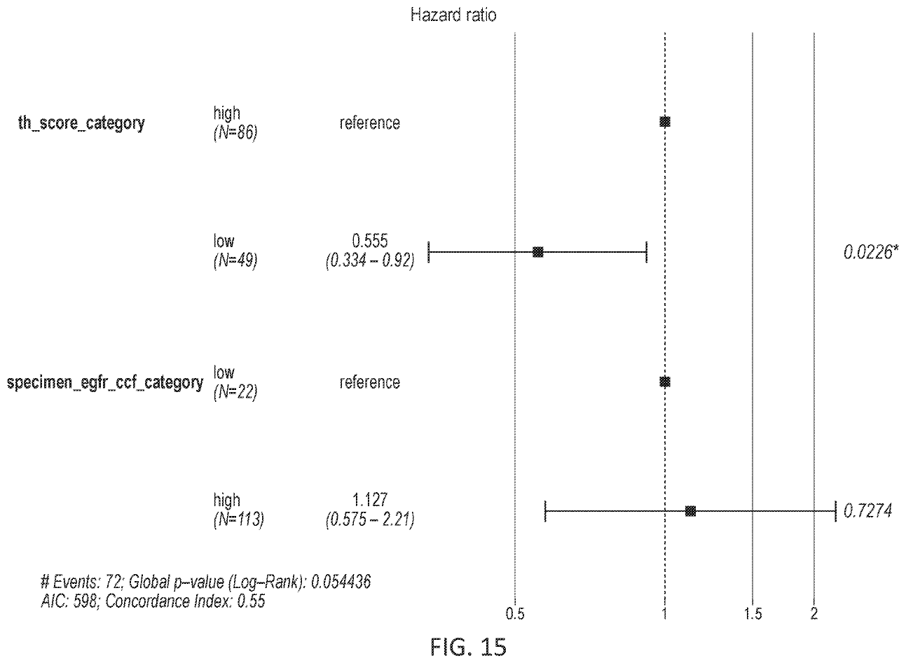

When the NSCLC patient cohort was broken down into four groups based on binary tumor heterogeneity score category and the CCF of the EGFR driver alteration (EGFR L858R or exon 19 deletion), an interesting pattern emerged. Patients with a low score and high EGFR CCF had the highest mPFS at 16.01 months, while patients with a high score and low EGFR CCF had the lowest mPFS at 6.54 months (see FIG. 14 and FIG. 15). Patient specimens for which the EGFR driver alteration is clonal and for which the specimen doesn't include many other competing alterations (CCF-wise) seem to respond the best to Osimertinib targeted therapy, although this correlation was not significant—probably because of the skewed distribution of the EGFR driver alteration's clonality, thereby affecting the numbers in the sub-grouping of patients. It is expected that EGFR driver alterations are always clonal in NSCLC. Here we observed that 113 of the 135 patients had a clonal EGFR alteration.

FIG. 14 provides a plot of progression free survival (PFS) probability from start of 1^{st} line Osimertinib treatment for the cohort stratified by a binary tumor heterogeneity score threshold of 1 and the underlying EGFR driver alteration's clonality. TH high group: tumor heterogeneity score >=1. TH low group: tumor heterogeneity score <1. EGFR high group: EGFR driver alteration CCF >=0.5. EGFR low group: EGFR driver alteration CCF <0.5. The lower panel in FIG. 14 provides table of the number of remaining patients at risk as a function of time following initiation of 1st Osimertinib treatment for each of the four score groups—TH High/EGFR Low, TH High/EGFR High, TH Low/EGFR Low, and TH Low/EGFR High.

FIG. 15 provides a plot of the results for a multivariate Cox proportional hazards regression model from start of 1^{st} line Osimertinib treatment for the cohort stratified by a binary tumor heterogeneity score (THS) threshold of 1 and the underlying EGFR driver alteration's clonality. The plot shows the hazard ratio for the TH high, TH low, CCF low, and CCF high groups. The log rank p-value indicates the significance of the result. AIC measures model quality (with a lower AIC indicating better model fit). The concordance index should be above 0.5 (higher values indicating better model fit).

Table 5 summarizes the median PFS for all four categories of patients for the Kaplan Meier curves seen in FIG. 14.

TABLE 5
Median PFS for all four categories of NSCLC cohort patients.

| Score | EGFR CCF | | | | | |
|---|---|---|---|---|---|---|
| category | category | N | n events | mPFS | LCI | UCI |
| Low | Low | 10 | 4 | NA | 9.8 | NA |
| Low | High | 39 | 18 | 16.01 | 14.93 | NA |
| High | Low | 12 | 6 | 6.54 | 3.2 | NA |
| High | High | 74 | 44 | 11.11 | 9.4 | 13.7 |

### EXEMPLARY IMPLEMENTATIONS

Exemplary implementations of the methods described herein include:
1. A method comprising:
   providing a plurality of nucleic acid molecules obtained from a sample from a subject;
   ligating one or more adapters onto one or more nucleic acid molecules from the plurality of nucleic acid molecules;
   amplifying the one or more ligated nucleic acid molecules from the plurality of nucleic acid molecules;
   capturing amplified nucleic acid molecules from the amplified nucleic acid molecules;
   sequencing, by a sequencer, the captured nucleic acid molecules to obtain sequence read data that represents the captured nucleic acid molecules;
   identifying a presence or absence of one or more variants in the sample based on the sequence read data;
   identifying a driver mutation in the sample based on the sequence read data;
   determining a tumor heterogeneity score for the sample based on the one or more variants identified as present in the sample; and
   comparing the tumor heterogeneity score to a threshold tumor heterogeneity score,
   wherein if the tumor heterogeneity score is less than or equal to the threshold tumor heterogeneity score, the subject is identified for treatment with a first anti-cancer agent selected at least in part based on the identified driver mutation, and
   wherein if the tumor heterogeneity score is greater than the threshold tumor heterogeneity score, the subject is identified for treatment with a second anti-cancer agent selected at least in part based on the identified driver mutation.
2. The method of clause 1, further comprising determining a clonality metric for the driver mutation, wherein if the tumor heterogeneity score is less than or equal to the threshold tumor heterogeneity score and the clonality metric is greater than or equal to a threshold clonality metric, the subject is identified for treatment with the first anti-cancer agent.
3. The method of clause 1, further comprising determining a clonality metric for the driver mutation, wherein if the tumor heterogeneity score is greater than the threshold tumor heterogeneity score and the clonality metric is less than a threshold clonality metric, the subject is identified for treatment with a second anti-cancer agent.
4. The method of any one of clauses 1 to 3, wherein if the tumor heterogeneity score is greater than the threshold tumor heterogeneity score, the subject is identified for tumor monitoring.
5. The method of any one of clauses 1 to 4, wherein the subject is suspected of having or is determined to have a cancer.
6. The method of any one of clauses 1 to 5, further comprising obtaining the sample from the subject.
7. The method of any one of clauses 1 to 6, wherein the sample comprises a tissue biopsy sample, a liquid biopsy sample, or a normal control.
8. The method of clause 7, wherein the sample is a liquid biopsy sample and comprises blood, plasma, cerebrospinal fluid, sputum, stool, urine, or saliva.
9. The method of clause 7, wherein the sample is a liquid biopsy sample and comprises circulating tumor cells (CTCs).
10. The method of clause 7, wherein the sample is a liquid biopsy sample and comprises cell-free DNA (cfDNA), circulating tumor DNA (ctDNA), or any combination thereof.
11. The method of any one of clauses 1 to 10, wherein the plurality of nucleic acid molecules comprises a mixture of tumor nucleic acid molecules and non-tumor nucleic acid molecules.
12. The method of clause 11, wherein the tumor nucleic acid molecules are derived from a tumor portion of a heterogeneous tissue biopsy sample, and the non-tumor nucleic acid molecules are derived from a normal portion of the heterogeneous tissue biopsy sample.
13. The method of clause 11, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.
14. The method of any one of clauses 1 to 13, wherein the one or more adapters comprise amplification primers, flow cell adaptor sequences, substrate adapter sequences, or sample index sequences.
15. The method of any one of clauses 1 to 14, wherein the captured nucleic acid molecules are captured from the amplified nucleic acid molecules by hybridization to one or more bait molecules.
16. The method of clause 15, wherein the one or more bait molecules comprise one or more nucleic acid molecules, each comprising a region that is complementary to a region of a captured nucleic acid molecule.
17. The method of any one of clauses 1 to 16, wherein amplifying nucleic acid molecules comprises performing a polymerase chain reaction (PCR) amplification technique, a non-PCR amplification technique, or an isothermal amplification technique.
18. The method of any one of clauses 1 to 17, wherein the sequencing comprises use of a massively parallel sequencing (MPS) technique, whole genome sequencing (WGS), whole exome sequencing, targeted sequencing, direct sequencing, or Sanger sequencing technique.
19. The method of clause 18, wherein the sequencing comprises massively parallel sequencing, and the massively parallel sequencing technique comprises next generation sequencing (NGS).
20. The method of any one of clauses 1 to 19, wherein the sequencer comprises a next generation sequencer.
21. The method of any one of clauses 1 to 20, further comprising generating, by the one or more processors, a report comprising a tumor heterogeneity score for the subject or a clonality metric for a driver mutation of the disease present in the sequence read data of the subject.
22. The method of clause 21, further comprising transmitting the report to a healthcare provider.
23. The method of clause 22, wherein the report is transmitted via a computer network or a peer-to-peer connection.
24. A method for identifying a subject having a cancer for treatment with an anti-cancer agent comprising:
   acquiring knowledge of a driver mutation in a sample obtained from the subject;
   identifying one or more variants present in the sample;
   determining a tumor heterogeneity score for the sample based on the one or more variants; and
   comparing the tumor heterogeneity score for the sample to a threshold tumor heterogeneity score,
   wherein if the tumor heterogeneity score is less than or equal to the threshold tumor heterogeneity score, the subject is identified for treatment with a first anti-cancer agent selected at least in part based on the knowledge of the driver mutation, and
   wherein if the tumor heterogeneity score is greater than the threshold tumor heterogeneity score, the subject is identified for treatment with a second anti-cancer agent selected at least in part based on the knowledge of the driver mutation.
25. A method of selecting a treatment for a subject having a cancer, the method comprising:
   acquiring knowledge of a driver mutation in a sample obtained from the subject;
   determining a tumor heterogeneity score for the sample obtained from the subject; and
   comparing the tumor heterogeneity score for the sample to a threshold tumor heterogeneity score,
   wherein if the tumor heterogeneity score that is less than or equal to the threshold tumor heterogeneity score, the subject is identified as one who may benefit from treatment with a first anti-cancer treatment selected at least in part based on the knowledge of the driver mutation; and
   wherein if the tumor heterogeneity score is greater than the threshold tumor heterogeneity score, the subject is identified as one who may benefit from treatment with a second anti-cancer treatment selected at least in part based on the knowledge of the driver mutation.
26. The method of clause 24 or clause 25, further comprising determining a clonality metric for the driver mutation, wherein if the tumor heterogeneity score is less than or equal to the threshold tumor heterogeneity score and the clonality metric is greater than or equal to a threshold clonality metric, the subject is identified for treatment with the first anti-cancer therapy or as one who may benefit from treatment with the first anti-cancer treatment.
27. The method of clause 24 or clause 25, further comprising determining a clonality metric for the driver mutation, wherein if the tumor heterogeneity score is greater than the threshold tumor heterogeneity score and the clonality metric is less than a threshold clonality metric, the subject is identified for treatment with a second anti-cancer therapy or as one who may benefit from treatment with the second anti-cancer treatment.
28. The method of any one of clauses 24 to 27, wherein if the tumor heterogeneity score is greater than the threshold tumor heterogeneity score, the subject is identified for tumor monitoring.
29. The method of clause 28, wherein the tumor monitoring comprises follow-up tumor sequencing within 3, 6, 9, or 12 months.
30. The method of clause 28 or clause 29, wherein the tumor monitoring comprises having a CT scan of the subject performed within 3, 6, 9, or 12 months.
31. A method of identifying one or more treatment options for a subject having a cancer, the method comprising:
   a) acquiring knowledge of a driver mutation in a sample obtained from the subject;
   b) determining a tumor heterogeneity score for the sample obtained from the subject; and
   c) generating a report comprising one or more treatment options identified for the subject that are selected at least in part based on the knowledge of the driver mutation, wherein a tumor heterogeneity score that is less than or equal to a corresponding threshold tumor heterogeneity score identifies the subject as one who may benefit from that treatment option.
32. The method of clause 31, further comprising determining a clonality metric for the driver mutation and generating the report comprising one or more treatment options identified for the subject, wherein a tumor heterogeneity score that is less than or equal to a corresponding threshold tumor heterogeneity score for each of one or more candidate treatment options and a clonality metric that is greater than or equal to a threshold clonality metric for each of the one or more candidate treatment options identifies the subject as one who may benefit from that treatment option.
33. The method of clause 31 or clause 32, wherein if the tumor heterogeneity score is greater than the threshold tumor heterogeneity score, the subject is identified for tumor monitoring.
34. A method of stratifying a subject with a cancer for treatment comprising:
   acquiring knowledge of a driver mutation in a sample obtained from the subject;
   determining a tumor heterogeneity score for the sample obtained from the subject; and
   a) if the tumor heterogeneity score is less than or equal to a threshold tumor heterogeneity score, identifying the subject as a candidate for receiving a first anti-cancer therapy selected at least in part based on the knowledge of the driver mutation; or
   b) if the tumor heterogeneity score is greater than the threshold tumor heterogeneity score, identifying the subject as a candidate for receiving a second anti-cancer therapy selected at least in part based on the knowledge of the driver mutation.
35. The method of clause 34, further comprising determining a clonality metric for the driver mutation; and
   c) if the tumor heterogeneity score is less than or equal to a threshold tumor heterogeneity score for a first therapy and the clonality metric is greater than or equal to a threshold clonality metric for the first anti-cancer therapy, identifying the subject as a candidate for receiving the first anti-cancer therapy; or
   d) if the tumor heterogeneity score is greater than the threshold tumor heterogeneity score for the first therapy and the clonality metric is less than the threshold clonality metric for the first anti-cancer therapy, identifying the subject as a candidate for receiving a second anti-cancer therapy.
36. The method of clause 34 or clause 35, wherein if the tumor heterogeneity score is greater than the threshold tumor heterogeneity score, the subject is identified for tumor monitoring.
37. A method for treating a subject having a cancer, the method comprising:
   a) acquiring knowledge of a driver mutation in a sample obtained from the subject;
   b) determining a tumor heterogeneity score for the sample obtained from the subject; and
   c) treating the subject with a first anti-cancer therapy selected at least in part based on the knowledge of the driver mutation if the tumor heterogeneity score is less than or equal to a threshold tumor heterogeneity score.
38. The method of clause 37, further comprising treating the subject with a second anti-cancer therapy selected at least in part based on the knowledge of the driver mutation if the tumor heterogeneity score is greater than the threshold tumor heterogeneity score.
39. The method of clause 37 or clause 38, further comprising determining a clonality metric for the driver mutation, and treating the subject with the first anti-cancer therapy if the tumor heterogeneity score is less than or equal to the threshold tumor heterogeneity score and the clonality metric is greater than or equal to a threshold clonality metric for the first anti-cancer therapy.
40. The method of clause 39, further comprising treating the subject with the second anti-cancer therapy if the tumor heterogeneity score is greater than the threshold tumor heterogeneity score and the clonality metric is less than the threshold clonality metric for the first anti-cancer therapy.
41. The method of any one of clauses 37 to 40, wherein if the tumor heterogeneity score is greater than the threshold tumor heterogeneity score, the subject is identified for tumor monitoring.
42. A method of predicting a duration of therapeutic response for a subject having a cancer, comprising:
   acquiring knowledge of a driver mutation in a sample obtained from the subject;
   determining a tumor heterogeneity score for the sample obtained from the subject; and
   comparing the tumor heterogeneity score for the sample to a threshold tumor heterogeneity score,
   wherein if the tumor heterogeneity score is less than or equal to the threshold tumor heterogeneity score, the subject is predicted to have a longer duration of therapeutic response to an anti-cancer therapy selected at least in part based on the knowledge of the driver mutation; and
   wherein if the tumor heterogeneity score is greater than the threshold tumor heterogeneity score, the subject is predicted to have a shorter therapeutic response to the anti-cancer therapy selected at least in part based on the knowledge of the driver mutation.
43. A method of predicting survival of a subject having a cancer comprising:
   acquiring knowledge of a driver mutation in a sample obtained from the subject;
   determining a tumor heterogeneity score for the sample obtained from the subject; and
   comparing the tumor heterogeneity score for the sample to a threshold tumor heterogeneity score,
   wherein if the tumor heterogeneity score is less than or equal to the threshold tumor heterogeneity score, the subject is predicted to have increased survival if treated with an anti-cancer therapy selected at least in part based on the knowledge of the driver mutation; and
   wherein if the tumor heterogeneity score is greater than the threshold tumor heterogeneity, the subject is predicted to have reduced survival if treated with the anti-cancer therapy selected at least in part based on the knowledge of the driver mutation.
44. A method of monitoring, evaluating, or screening a subject having a cancer, comprising acquiring knowledge of a driver mutation in a sample obtained from the subject; determining a tumor heterogeneity score for the sample; and comparing the tumor heterogeneity score to a threshold tumor heterogeneity score, wherein if the tumor heterogeneity score is less than or equal to the threshold tumor heterogeneity score, the subject is predicted to have increased survival when treated with an anti-cancer therapy selected at least in part based on the knowledge of the driver mutation, and wherein if the tumor heterogeneity score is greater than the threshold tumor heterogeneity score, the subject is predicted to have reduced survival when treated with the anti-cancer therapy selected at least in part based on the knowledge of the driver mutation.
45. The method of any one of clauses 42 to 44, wherein if the tumor heterogeneity score is greater than the threshold tumor heterogeneity score, the subject is identified for tumor monitoring.
46. The method of any one of clauses 43 to 45, wherein the increased survival is increased overall survival (OS), increased progression free survival (PFS), increased disease-free survival (DFS), increased objective response rate (ORR), increased time to tumor progression (TTP), increased time to treatment failure (TTF), increased durable complete response (DCR), or increased time to next treatment (TTNT).
47. The method of any one of clauses 43 to 46, wherein the increased survival is increased overall survival (OS).
48. The method of any one of clauses 43 to 46, wherein the increased survival is increased progression free survival (PFS).
49. The method of any one of clauses 24 to 48, wherein the determination of a tumor heterogeneity score is based on sequence read data derived from the sample obtained from the subject.
50. The method of clause 49, wherein the sequence read data is obtained by performing next-generation sequencing (NGS), massively parallel sequencing (MPS), whole genome sequencing (WGS), whole exome sequencing (WES), targeted sequencing, direct sequencing, or Sanger sequencing.
51. The method of clause 49 or clause 50, wherein the sequence read is obtained by performing next-generation sequencing (NGS).
52. The method of any one of clauses 24 to 51, wherein the sample comprises cells and/or nucleic acids from the cancer.
53. The method of any one of clauses 24 to 52, wherein the sample obtained from the subject comprises a single biopsy sample.
54. The method of any one of clauses 24 to 53, wherein the sample obtained from the subject comprises multiple biopsy samples.
55. The method of any one of clauses 24 to 54, wherein the sample comprises a tissue biopsy sample, a liquid biopsy sample, or a normal control.
56. The method of clause 55, wherein the sample comprises a tissue biopsy sample, and the tissue biopsy sample comprises a tumor biopsy, a tumor specimen, or a circulating tumor cell.
57. The method of clause 55, wherein the sample comprises a liquid biopsy sample, and the liquid biopsy sample comprises blood, serum, plasma, cerebrospinal fluid, sputum, stool, urine, or saliva.
58. The method of clause 57, wherein the cancer is non-small cell lung cancer (NSCLC), prostate cancer, ovarian cancer, breast cancer, melanoma, colorectal cancer, a cholangiocarcinoma, or prostate cancer.
59. The method of clause 55, wherein the sample comprises a liquid biopsy sample, and sequence read data for the subject is obtained by sequencing circulating tumor DNA (ctDNA) in the liquid biopsy sample.
60. The method of any one of clauses 24 to 59, wherein the cancer is bladder cancer, brain cancer, breast cancer, cervical cancer, colorectal cancer, dermatofibrosarcoma protuberans, an endocrine/neuroendocrine tumor, esophageal cancer, head and neck cancer, a gastrointestinal stromal tumor, a giant cell tumor, kidney cancer, leukemia, liver and bile duct cancer, lung cancer, lymphoma, a malignant mesothelioma, a microsatellite instability-high or mismatch repair-deficient solid tumor, multiple myeloma, a myelodysplastic/myeloproliferative disorder, a neuroblastoma, an ovarian epithelial/fallopian tube/primary peritoneal cancer, pancreatic cancer, a plexiform neurofibroma, prostate cancer, skin cancer, a soft tissue sarcoma, a solid tumor having a high tumor mutational burden (TMB-H), a solid tumor comprising a neurotrophic tyrosine receptor kinase (NTRK) gene fusion, stomach (gastric) cancer, a systemic mastocytosis, a thyroid cancer, or any combination thereof.
61. The method of any one of clauses 24 to 60, wherein the cancer is acute lymphoblastic leukemia (Philadelphia chromosome positive), acute lymphoblastic leukemia (precursor B-cell), acute myeloid leukemia (FLT3+), acute myeloid leukemia (with IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), colorectal cancer (MSI-H/dMMR), cryopyrin-associated periodic syndromes, cutaneous T-cell lymphoma, dermatofibrosarcoma protuberans, diffuse large B-cell lymphoma, fallopian tube cancer, follicular B-cell non-Hodgkin lymphoma, follicular lymphoma, gastric cancer, gastric cancer (HER2+), gastroesophageal junction (GEJ) adenocarcinoma, gastrointestinal stromal tumors, gastrointestinal stromal tumor (KIT+), giant cell tumor of the bone, glioblastoma, granulomatosis with polyangiitis, head and neck squamous cell carcinoma, hepatocellular carcinoma, Hodgkin lymphoma, juvenile idiopathic arthritis, lupus erythematosus, mantle cell lymphoma, medullary thyroid cancer, melanoma, melanoma (with BRAF V600 mutation), melanoma (with BRAF V600E or V600K mutation), merkel cell carcinoma, multicentric Castleman's disease, multiple hematologic malignancies including Philadelphia chromosome-positive ALL and CML, multiple myeloma, myelofibrosis, non-Hodgkin's lymphoma, nonresectable subependymal giant cell astrocytoma associated with tuberous sclerosis, non-small cell lung cancer, non-small cell lung cancer (ALK+), non-small cell lung cancer (PD-L1+), non-small cell lung cancer (with ALK fusion or ROS1 gene alteration), non-small cell lung cancer (with BRAF V600E mutation), non-small cell lung cancer (with EGFR exon 19 deletions or exon 21 substitution (L858R) mutations), non-small cell lung cancer (with EGFR T790M mutation), ovarian cancer, ovarian cancer (with BRCA mutation), pancreatic cancer, pancreatic, gastrointestinal, or lung origin neuroendocrine tumor, pediatric neuroblastoma, peripheral T-cell lymphoma, peritoneal cancer, prostate cancer, renal cell carcinoma, rheumatoid arthritis, small lymphocytic lymphoma, soft tissue sarcoma, solid tumors (MSI-H/dMMR), squamous cell cancer of the head and neck, squamous non-small cell lung cancer, thyroid cancer, thyroid carcinoma, urothelial cancer, urothelial carcinoma, or Waldenstrom's macroglobulinemia.
62. The method of any one of clauses 24 to 61, wherein the cancer is a solid tumor.
63. The method of any one of clauses 24 to 62, wherein the cancer comprises a driver mutation or variant in an ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene, or any combination thereof.
64. The method of any one of clauses 24 to 63, wherein the cancer comprises a driver mutation or variant in the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB gene, or any combination thereof.
65. The method of any one of clauses 24 to 64, wherein the cancer comprises a driver mutation in the ALK, ATM, BARD1, BRAF, BRCA1, BRCA2, BRIP1, CDK12, CHEK1, CHEK2, EGFR, ERBB2 (HER2), FANCL, FGFR2, KRAS, MET, NRAS, NTRK1, NTRK2, NTRK3, PIK3CA, PALB2, RAD51B, RAD51C, RAD51D, RAD54L, OR RET gene.
66. The method of any one of clauses 24 to 65, wherein the cancer comprises an epidermal growth factor receptor (EGFR) driver mutation.
67. The method of clause 66, wherein the EGFR driver mutation comprises an EGFR exon 21 L858R mutation, an EGFR exon 19 deletion, an EGFR T790M mutation, or an EGFR amplification.
68. The method of any one of clauses 24 to 65, wherein the cancer comprises an ALK driver mutation.
69. The method of clause 68, wherein the ALK driver mutation comprises an ALK rearrangement.
70. The method of any one of clauses 24 to 65, wherein the cancer comprises a BRAF driver mutation.
71. The method of clause 70, wherein the BRAF driver mutation comprises a BRAF V600E mutation or a BRAF V600K mutation.
72. The method of any one of clauses 24 to 65, wherein the cancer comprises a MET driver mutation.
73. The method of clause 72, wherein the MET driver mutation comprises a MET single nucleotide variant or a MET indel that leads to MET exon 14 skipping.
74. The method of any one of clauses 24 to 65, wherein the cancer comprises an ERBB2 (HER2) driver mutation.
75. The method of clause 74, wherein the ERBB2 (HER2) driver mutation comprises an ERBB2 (HER2) amplification.
76. The method of any one of clauses 24 to 65, wherein the cancer comprises a PIK3CA driver mutation.
77. The method of clause 76, wherein the PIK3CA driver mutation comprises a PIK3CA C420R mutation, a PIK3CA E542K mutation, a PIK3CA E545A mutation, a PIK3CA E545D mutation, a PIK3CA E545G mutation, a PIK3CA E545K mutation, a PIK3CA Q546E mutation, a PIK3CA Q546R mutation, a PIK3CA H1047L mutation, a PIK3CA H1047R mutation, or a PIK3CA H1047Y mutation.
78. The method of any one of clauses 24 to 65, wherein the cancer comprises a KRAS driver mutation.
79. The method of clause 78, wherein the KRAS driver mutation comprises a KRAS mutation in codon 12, codon 13, exon 2, exon 3, or exon 4.
80. The method of any one of clauses 24 to 65, wherein the cancer comprises a NRAS driver mutation.
81. The method of clause 80, wherein the NRAS driver mutation comprises a mutation in exon 2, exon 3, or exon 4.
82. The method of any one of clauses 24 to 65, wherein the cancer comprises a BRCA1 driver mutation.
83. The method of any one of clauses 24 to 65, wherein the cancer comprises a BRCA2 driver mutation.
84. The method of any one of clauses 24 to 65, wherein the cancer comprises an ATM driver mutation.
85. The method of any one of clauses 24 to 65, wherein the cancer comprises an FGFR2 driver mutation.
86. The method of clause 85, wherein the FGFR2 driver mutation comprises an FGFR2 fusion or an FGFR2 rearrangement.
87. The method of any one of clauses 24 to 65, wherein the cancer comprises a driver mutation in a homologous recombination repair (HRR) gene.
88. The method of clause 87, wherein the driver mutation in the homologous recombination repair (HRR) gene comprises a BRCA1, BRCA2, ATM, BARD1, BRIP1, CDK12, CHEK1, CHEK2, FANCL, PALB2, RAD51B, RAD51C, RAD51D, or RAD54L mutation.
89. The method of any one of clauses 24 to 65, wherein the cancer comprises an NTRK1 driver mutation.
90. The method of clause 89, wherein the NTRK1 driver mutation comprises an NTRK1 fusion.
91. The method of any one of clauses 24 to 65, wherein the cancer comprises an NTRK2 driver mutation.
92. The method of clause 91, wherein the NTRK2 driver mutation comprises an NTRK2 fusion.
93. The method of any one of clauses 24 to 65, wherein the cancer comprises an NTRK3 driver mutation.
94. The method of clause 93, wherein the NTRK3 driver mutation comprises an NTRK3 fusion.
95. The method of any one of clauses 24 to 65, wherein the cancer comprises a RET driver mutation.
96. The method of clause 95, wherein the RET driver mutation comprises a RET fusion.
97. The method of any one of clauses 24 to 96, wherein the threshold tumor heterogeneity score ranges in value from 0.1 to 20.
98. The method of any one of clauses 24 to 97, wherein the threshold tumor heterogeneity score ranges in value from 0.5 to 2.0.
99. The method of any one of clauses 26 to 98, wherein the threshold clonality metric ranges in value from 0.1 to 0.9.
100. The method of any one of clauses 26 to 99, wherein the threshold clonality metric ranges in value from 0.4 to 0.6.
101. The method of any one of clauses 24 to 100, wherein the first anti-cancer agent, second anti-cancer agent, first anti-cancer treatment, second anti-cancer treatment, candidate treatment, first anti-cancer therapy, second anti-cancer therapy, or selected anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aligopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubega), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.
102. The method of any one of clauses 24 to 101, wherein the first anti-cancer agent, second anti-cancer agent, first anti-cancer treatment, second anti-cancer treatment, candidate treatment, first anti-cancer therapy, second anti-cancer therapy, or selected anti-cancer therapy comprises Gilotrif® (afatinib), Iressa® (gefitinib), Tagrisso® (osimertinib), Tarceva® (erlotinib), Alecensa® (alectinib), Alunbrig® (brigatinib), Xalkori® (crizotinib), Zykadia® (ceritinib), Tafinlar® (dabrafenib), Mekinist® (trametinib), Tabrecta® (capmatinib), Tecentriq® (atezolizumab), Cotellic® (cobimetinib), Zelboraf® (vemurafenib), Herceptin® (trastuzumab), Kadcyla® (ado-trastuzumabemtansine), Perjeta® (pertuzumab), Piqray® (alpelisib), Erbitux® (cetuximab), Vectibix® (panitumumab), Lynparza® (olaparib), Rubraca® (rucaparib), Pemazyre® (pemigatinib), Truseltiq™ (infigratinib), Keytruda® (pembrolizumab), Vitrakvi® (larotrectinib), Mekinist® (Trametinib), or Retevmo (selpercatinib).
103. The method of any one of clauses 24 to 102, wherein the first anti-cancer agent, second anti-cancer agent, first anti-cancer treatment, second anti-cancer treatment, candidate treatment, first anti-cancer therapy, second anti-cancer therapy, or selected anti-cancer therapy comprises an EGFR tyrosine kinase inhibitor.
104. The method of any one of clauses 24 to 103, wherein the first anti-cancer agent, second anti-cancer agent, first anti-cancer treatment, second anti-cancer treatment, candidate treatment, first anti-cancer therapy, second anti-cancer therapy, or selected anti-cancer therapy comprises Osimertinib.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

What is claimed is:

1. A method comprising:
providing a plurality of nucleic acid molecules obtained from a sample from a subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain sequence read data that represents the captured nucleic acid molecules;
identifying a presence or absence of one or more variants in the sample based on the sequence read data;
identifying a driver mutation in the sample based on the sequence read data;
determining a tumor heterogeneity score for the sample based on the one or more variants identified as present in the sample; and
comparing the tumor heterogeneity score to a threshold tumor heterogeneity score,
wherein if the tumor heterogeneity score is less than or equal to the threshold tumor heterogeneity score, the subject is identified for treatment with a first anti-cancer agent selected at least in part based on the identified driver mutation, and
wherein if the tumor heterogeneity score is greater than the threshold tumor heterogeneity score, the subject is identified for treatment with a second anti-cancer agent selected at least in part based on the identified driver mutation.

2. The method of claim 1, further comprising determining a clonality metric for the driver mutation, wherein if the tumor heterogeneity score is less than or equal to the threshold tumor heterogeneity score and the clonality metric is greater than or equal to a threshold clonality metric, the subject is identified for treatment with the first anti-cancer therapy or as one who may benefit from treatment with the first anti-cancer agent.

3. The method of claim 1, further comprising determining a clonality metric for the driver mutation, wherein if the tumor heterogeneity score is greater than the threshold tumor heterogeneity score and the clonality metric is less than a threshold clonality metric, the subject is identified for treatment with a second anti-cancer therapy or as one who may benefit from treatment with the second anti-cancer agent.

4. The method of claim 1, wherein if the tumor heterogeneity score is greater than the threshold tumor heterogeneity score, the subject is identified for tumor monitoring.

5. The method of claim 4, wherein the tumor monitoring comprises follow-up tumor sequencing within 3, 6, 9, or 12 months.

6. The method of claim 4, wherein the tumor monitoring comprises having a CT scan of the subject performed within 3, 6, 9, or 12 months.

7. The method of claim 1, wherein the sample obtained from the subject comprises a single biopsy sample.

8. The method of claim 1, wherein the sample comprises a liquid biopsy sample, and the liquid biopsy sample comprises blood, serum, plasma, cerebrospinal fluid, sputum, stool, urine, or saliva.

9. The method of claim 1, wherein the sample comprises a liquid biopsy sample, and sequence read data for the subject is obtained by sequencing circulating tumor DNA (ctDNA) in the liquid biopsy sample.

10. A method for identifying a subject having a cancer for treatment with an anti-cancer agent comprising:
acquiring knowledge of a driver mutation in a sample obtained from the subject;
identifying one or more variants present in the sample;
determining a tumor heterogeneity score for the sample based on the one or more variants; and
comparing the tumor heterogeneity score for the sample to a threshold tumor heterogeneity score,
wherein if the tumor heterogeneity score is less than or equal to the threshold tumor heterogeneity score, the subject is identified for treatment with a first anti-cancer agent selected at least in part based on the knowledge of the driver mutation, and
wherein if the tumor heterogeneity score is greater than the threshold tumor heterogeneity score, the subject is identified for treatment with a second anti-cancer agent selected at least in part based on the knowledge of the driver mutation.

11. The method of claim 10, wherein if the tumor heterogeneity score is greater than the threshold tumor heterogeneity score, the subject is identified for tumor monitoring.

12. The method of claim 10, wherein the cancer is non-small cell lung cancer (NSCLC), prostate cancer, ovarian cancer, breast cancer, melanoma, colorectal cancer, a cholangiocarcinoma, or prostate cancer.

13. The method of claim 10, wherein the cancer comprises an epidermal growth factor receptor (EGFR) driver mutation, an ALK driver mutation, BRAF driver mutation, a MET driver mutation, an ERBB2 (HER2) driver mutation, a PIK3CA driver mutation, a KRAS driver mutation, a NRAS driver mutation, a BRCA1 driver mutation, a BRCA2 driver mutation, an ATM driver mutation, an FGFR2 driver mutation, an NTRK1 driver mutation, an NTRK2 driver mutation, an NTRK3 driver mutation, a RET driver mutation, or a driver mutation in a homologous recombination repair (HRR) gene.

14. The method of claim 10, wherein the cancer comprises a driver mutation in a homologous recombination repair (HRR) gene, and the driver mutation in the homologous recombination repair (HRR) gene comprises a BRCA1, BRCA2, ATM, BARD1, BRIP1, CDK12, CHEK1, CHEK2, FANCL, PALB2, RAD51B, RAD51C, RAD51D, or RAD54L mutation.

15. The method of claim 10, wherein the first anti-cancer agent or second anti-cancer agent comprises an EGFR tyrosine kinase inhibitor.

16. The method of claim 10, wherein the first anti-cancer agent or second anti-cancer agent comprises Osimertinib.

17. A method of identifying one or more treatment options for a subject having a cancer, the method comprising:
a) acquiring knowledge of a driver mutation in a sample obtained from the subject;
b) determining a tumor heterogeneity score for the sample obtained from the subject; and
c) generating a report comprising one or more treatment options identified for the subject that are selected at least in part based on the knowledge of the driver mutation, wherein a tumor heterogeneity score that is less than or equal to a corresponding threshold tumor heterogeneity score identifies the subject as one who may benefit from that treatment option.

18. The method of claim 17, wherein if the tumor heterogeneity score is greater than the threshold tumor heterogeneity score, the subject is identified for tumor monitoring.

19. The method of claim 17, further comprising determining a clonality metric for the driver mutation and generating the report comprising one or more treatment options identified for the subject, wherein a tumor heterogeneity score that is less than or equal to a corresponding threshold tumor heterogeneity score for each of one or more candidate treatment options and a clonality metric that is greater than or equal to a threshold clonality metric for each of the one or more candidate treatment options identifies the subject as one who may benefit from that treatment option.

20. The method of claim 17, further comprising determining a clonality metric for the driver mutation; and
d) if the tumor heterogeneity score is less than or equal to a threshold tumor heterogeneity score for a first anti-cancer therapy and the clonality metric is greater than or equal to a threshold clonality metric for the first anti-cancer therapy, identifying the subject as a candidate for receiving the first anti-cancer therapy; or
e) if the tumor heterogeneity score is greater than the threshold tumor heterogeneity score for the first anti-cancer therapy and the clonality metric is less than the threshold clonality metric for the first anti-cancer therapy, identifying the subject as a candidate for receiving a second anti-cancer therapy.
